SEQUENCE LISTING (42 sequences)

SEQ ID NO: 1 - protein, 15 aa, Artificial
  The peptide was designed and synthesized.
EKAKYEAYKAAAAAA

SEQ ID NO: 2 - protein, 15 aa, Artificial
  The peptide was designed and synthesized.
EKPKYEAYKAAAAPA

SEQ ID NO: 3 - protein, 15 aa, Artificial
  The peptide was designed and synthesized.
EKPKFEAYKAAAAPA

SEQ ID NO: 4 - protein, 15 aa, Artificial
  The peptide was designed and synthesized.
EKAKEEAYKAAAAAA

SEQ ID NO: 5 - protein, 15 aa, Artificial
  The peptide was designed and synthesized.
EKPKVEAYKAAAAPA

SEQ ID NO: 6 - protein, 15 aa, Artificial
  The peptide was designed and synthesized.
EKPKEEAFKAAAAPA

SEQ ID NO: 7 - protein, 15 aa, Artificial
  The peptide was designed and synthesized.
EKAKFEAFKAAAAAA

SEQ ID NO: 8 - protein, 17 aa, Artificial
  The peptide was designed and synthesized.
APEKAKFEAFKAAAAPA

SEQ ID NO: 9 - protein, 17 aa, Artificial
  The peptide was designed and synthesized.
APEKAKFEAYKAAAAPA

SEQ ID NO: 10 - protein, 17 aa, Artificial
  The peptide was designed and synthesized.
APEKAKVEAFKAAAAPA

SEQ ID NO: 11 - protein, 15 aa, Artificial
  The peptide was designed and synthesized.
EAKKYEAYKAAAAAA

SEQ ID NO: 12 - protein, 15 aa, Artificial
  The peptide was designed and synthesized.
EAPKFEAYKAAAAPA

SEQ ID NO: 13 - protein, 15 aa, Artificial
  The peptide was designed and synthesized.
EAPKVEAYKAAAAPA

SEQ ID NO: 14 - protein, 15 aa, Artificial
  The peptide was designed and synthesized.
EAPKFEAFKAAAAPA

SEQ ID NO: 15 - protein, 17 aa, Artificial
  The peptide was designed and synthesized.
APEAKKFEAFKAAAAPA

SEQ ID NO: 16 - protein, 17 aa, Artificial
  The peptide was designed and synthesized.
APEAKKFEAYKAAAAPA

SEQ ID NO: 17 - protein, 17 aa, Artificial
  The peptide was designed and synthesized.
APEAKKVEAFKAAAAPA

SEQ ID NO: 18 - protein, 9 aa, Artificial
  The peptide was designed and synthesized.
EAKKVEAFK

SEQ ID NO: 19 - protein, 5 aa, Artificial
  The peptide was designed and synthesized.
YEAYK

SEQ ID NO: 20 - protein, 5 aa, Artificial
  The peptide was designed and synthesized.
FEAYK

SEQ ID NO: 21 - protein, 5 aa, Artificial
  The peptide was designed and synthesized.
EEAYK

SEQ ID NO: 22 - protein, 5 aa, Artificial
  The peptide was designed and synthesized.
VEAYK

SEQ ID NO: 23 - protein, 5 aa, Artificial
  The peptide was designed and synthesized.
EEAFK

SEQ ID NO: 24 - protein, 5 aa, Artificial
  The peptide was designed and synthesized.
FEAFK

SEQ ID NO: 25 - protein, 5 aa, Artificial
  The peptide was designed and synthesized.
VEAFK

SEQ ID NO: 26 - protein, 5 aa, Artificial
  The peptide was designed and synthesized.
YEAFK

SEQ ID NO: 27 - protein, 9 aa, Artificial
  The peptide was designed and synthesized.
EKAKYEAYK

SEQ ID NO: 28 - protein, 9 aa, Artificial
  The peptide was designed and synthesized.
EKPKYEAYK

SEQ ID NO: 29 - protein, 9 aa, Artificial
  The peptide was designed and synthesized.
EKPKFEAYK

SEQ ID NO: 30 - protein, 9 aa, Artificial
  The peptide was designed and synthesized.
EKAKEEAYK

SEQ ID NO: 31 - protein, 9 aa, Artificial
  The peptide was designed and synthesized.
EKPKVEAYK

SEQ ID NO: 32 - protein, 9 aa, Artificial
  The peptide was designed and synthesized.
EKPKEEAFK

SEQ ID NO: 33 - protein, 9 aa, Artificial
  The peptide was designed and synthesized.
EKAKFEAFK

SEQ ID NO: 34 - protein, 9 aa, Artificial
  The peptide was designed and synthesized.
EKAKFEAYK

SEQ ID NO: 35 - protein, 9 aa, Artificial
  The peptide was designed and synthesized.
EKAKVEAFK

SEQ ID NO: 36 - protein, 9 aa, Artificial
  The peptide was designed and synthesized.
EAKKYEAYK

SEQ ID NO: 37 - protein, 9 aa, Artificial
  The peptide was designed and synthesized.
EAPKFEAYK

SEQ ID NO: 38 - protein, 9 aa, Artificial
  The peptide was designed and synthesized.
EAPKVEAYK

SEQ ID NO: 39 - protein, 9 aa, Artificial
  The peptide was designed and synthesized.
EAPKFEAFK

SEQ ID NO: 40 - protein, 9 aa, Artificial
  The peptide was designed and synthesized.
EAKKFEAFK

SEQ ID NO: 41 - protein, 9 aa, Artificial
  The peptide was designed and synthesized.
EAKKFEAYK

SEQ ID NO: 42 - protein, 15 aa, Homo sapiens
ENPVVHFFKNIVTPR

(19) United States
(12) Patent Application Publication
(10) Pub. No.: US 2007/0264229 A1
(43) Pub. Date: Nov. 15, 2007

(54) Peptides for Treatment of Autoimmune Disease

(76) Inventor: Jack L. Strominger, Cambridge, MA (US)

Correspondence Address: LAWSON & WEITZEN, LLP, 88 BLACK FALCON AVE, SUITE 345, BOSTON, MA, 02210, US

(21) Appl. No.: 11/662,697

(22) PCT Filed: Sep. 13, 2005
(86) PCT No.: PCT/US05/32412

§ 371 Date: Mar. 13, 2007

Related U.S. Application Data
(60) Provisional application No. 60/609,496, filed on Sep. 13, 2004.

(51) Int. Cl. A61K 38/00; A61K 38/21; A61K 39/00; A61K 39/395; A61K 45/00; A61P 21/00; C07K 14/00

(52) U.S. Cl. 424/85.2; 424/130.1; 424/185.1; 424/85.1; 424/85.5; 514/12; 530/300

(57) ABSTRACT

Peptides are provided that both inhibit more strongly than synthetic amino acid polymers the binding of MBP85-99 to HLA-DR2 (DRA/DRB1*1501), and inhibit interleukin-2 production by two MBP85-99-specific HLA-DR2-restricted T cells. Peptide 15mers J5, J3, and J2 suppress MBP85-99-induced EAE in humanized mice and proteolipid protein 139-151-induced EAE in SJL/J mice. Spleen and lymph node cultures stimulated with these peptides produced large amounts of Th2 cytokines (IL-4 and IL-10), and adoptive transfer of T cell lines established from those cultures suppressed disease induction. These peptides provide specific, non-random sequences that are as effective as random copolymers in suppressing EAE in several murine models, and may be useful in treatment of MS.

### FIELD OF THE INVENTION

Peptide compositions and methods of use are provided for autoimmune diseases, such as multiple sclerosis, in a subject.

### BACKGROUND

Multiple sclerosis (MS) is an autoimmune inflammatory disease of the central nervous system (CNS) caused by lymphocyte and macrophage infiltrations into the white matter resulting in demyelination. The disease is commonly observed in young Caucasian adults with Northern European ancestry and is associated with the HLA-DR2 haplotype DRA*0101, DRB1*1501, DQA1*0102, DQB1*0602 (Spielman, R. S. et al. 1982. Epidemiol Rev 4:45; Hillert, J. et al. 1994. J Neuroimmunol 50:95; Oksenberg, J. R. et al. 1993. Jama 270:2362). Myelin basic protein (MBP) is thought to be one of the major target antigens in the pathogenesis of MS. Particularly, T cell reactivity to the immunodominant MBP 85-99 epitope is found in subjects carrying HLA-DR2, a genetic marker for susceptibility to MS. HLA-DR2-restricted MBP-specific T cells are clonally expanded and activated in MS patients (Wucherpfennig, K. W. et al. 1991. Immunol Today 12:277; Markovic-Plese, S. et al. 1995. J Immunol 155:982; Kerlero de Rosbo, N. et al. 1997. Eur J Immunol 27:3059; Tsuchida, T. et al. 1994. Proc Natl Acad Sci USA 91:10859; Illes, Z. et al. 1999. J Immunol 162:1811; Allegretta, M. et al. 1990. Science 247:718). Furthermore, HLA-DR2/MBP 85-99 complexes have been detected in the CNS plaques of these patients (Krogsgaard, M. et al. 2000. J Exp Med 191:1395). Critical residues for binding to HLA-DR2 and for TCR recognition of the MBP 85-99 epitope have been defined (Wucherpfennig, K. W. et al. 1994. J Exp Med 179:279; Smith, K. J. et al. 1998. J Exp Med 188:1511).

MS has been linked to the autoimmune response of T cells to myelin self-antigens presented by HLA-DR2 with which MS is genetically associated. Myelin basic protein (MBP) is a major candidate autoantigen in this disease. Its immunodominant epitope, MBP85-99, forms a complex with HLA-DR2. Copolymer 1 (Cop1, Copaxone®, Glatiramer Acetate, poly(Y, E, A, K) n), a random amino acid copolymer [poly (Y,E,A,K)n or YEAK] as well as two new synthetic copolymers [poly (F,Y,A,K)n or FYAK, and poly (V,W,A,K)n or VWAK] also form complexes with HLA-DR2 (DRA/DRB1*1501) and compete with MBP85-99 for binding.

Several therapeutic approaches to MS have been previously attempted utilizing cytokines, copolymers, dimers of class II MHC-peptide complexes, peptide antigens that induce anergy, vaccination with TCR and an altered peptide ligand (APL; Fridkis-Hareli, M. et al. 2001 Hum Immunol 62:753; Gaur, A. et al. 1992. Science 258:1491; Leonard, J. P. et al. 1996. Ann NY Acad Sci 795:216; Nicholson, L. B. et al. 1995. Immunity 3:397; Fridkis-Hareli, M. et al. 2002. J Clin Invest 109:1635; Ruiz, P. J. 2001. J Immunol 167:2688; Goodkin, D. E. et al. 2000. Neurology 54:1414). Many of these studies aimed to interfere with the MBP85-99-specific T cell recognition and/or the T cell response to deviate from the Th1 to the Th2 phenotype. Copolymer 1 Cop1, Copaxone®, Glatiramer Acetate, poly(Y, E, A, K)n, the only approved drug known to reduce MBP-specific T cell responses, reduces the relapse rate by 30% in relapsing-remitting forms of MS (Teitelbaum, D. et al. 1971. Eur J Immunol 1:242; Teitelbaum, D. et al. 1973. Eur J Immunol 3:273; Teitelbaum, D. et al. 1974. Clin Immunol Immunopathol 3:256; Aharoni, R. et al. 1993. Eur J Immunol 23:17; Bornstein, M. B. et al. 1987. N Engl J Med 317:408; Johnson, K. P. et al. 1995. Neurology 45:1268; Johnson, K. P. et al. 1998. Neurology 50:701). Cop1 binds to several human HLA-DR molecules, including HLA-DR2, and thus blocks modestly the presentation of MBP85-99 (Fridkis-Hareli, M. et al. 1998. J Immunol 160:4386; Fridkis-Hareli, M. et al. 1999. J Immunol 162:4697). Additional therapeutic agents to treat MS are needed.

### SUMMARY

An embodiment of the present invention is a composition having a peptide comprising an amino acid sequence selected from the group consisting of: YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26). The peptide comprising any of these sequences is in some embodiments at least six amino acids in length, or at least 15 amino acids in length.

Another embodiment is a composition having a peptide comprising an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17). Yet another embodiment of the present invention is a composition having a peptide comprising an amino acid sequence selected from the group consisting of: EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), EAKKFEAYK (SEQ ID NO: 41), and EAKKVEAFK (SEQ ID NO: 17).

Additional embodiments include, for any of the above compositions, related peptides for which the amino acid sequence further contains at least one alanine residue (A) at a terminus of the peptide. For example, the terminus is an amino terminus. Alternatively, the terminus is a carboxy terminus. Further related embodiments for any of these compositions are peptides in which the amino acid sequence further contains a plurality of A residues at a terminus of the peptide. In related embodiments, the plurality of A residues further contains at least one proline (P) residue at a position that is penultimate to the terminus of the peptide.

Another embodiment a composition having a peptide consisting essentially of an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), PEKAKFEAYKAAAAPA (SEQ ID NO: 9), APEKAKVEAFKAAAAPA (SEQ ID NO: 10), AKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17).

The invention in another embodiment provides a composition having a peptide with an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), PEKAKFEAYKAAAAPA (SEQ ID NO: 9), APEKAKVEAFKAAAAPA (SEQ ID NO: 10), AKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17).

Combination compositions are provided in further embodiments of the present invention. Accordingly, an embodiment of the invention provides a composition comprising a plurality of peptides according to any of the compositions above. Further, in related embodiments the invention provides for any of the above peptides, a terminal amino acid residue that is substituted, i.e., a terminal amino acid that is a chemical derivative of the original amino acid at the terminus. As used herein, the term “substituted” refers to a chemical derivative of the amino acid found in the parent amino acid sequence at that residue location. For example, in certain embodiments of the peptides provided herein, an amino terminal amino acid residue is substituted. Alternatively, a carboxy terminal amino acid residue is substituted. Typically, the substituted terminal amino acid is acetylated, methylated, formylated, or t-butyloxylated, for example, the amino terminal amino acid residue is acetylated. Typically, the carboxy terminal amino acid residue is methylated or amidated.

In further embodiments of peptides provided herein, an internal amino acid residue is substituted, for example, the internal amino acid residue is a lysine (K), and is substituted at the ε-amino group of the lysine. In other embodiments of peptides provided herein, at least one peptide bond is substituted by a non-peptide bond. For example, the non-peptide bond is selected from the group consisting of: —CH₂NH—, —CH₂S—, —CH₂CH₂—, —CH═CH—, —COCH₂—, —CH(OH)CH₂—, and —CH₂SO—. In certain embodiments of the compositions, at least one amino acid residue is substituted by an amino acid residue analog. An exemplary amino acid residue analog is selected from the group consisting of: a D-amino acid, an alkylated amino acid, a halogenated amino acid, and an amino acid substituted with a non-naturally occurring side chain.

The invention in another embodiment provides a pharmaceutical composition having at least one peptide from the group of peptides having amino acid sequences: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), APEAKKVEAFKAAAAPA (SEQ ID NO: 17), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26) in an effective dose. For any of these pharmaceutical compositions, the peptide is at least 6-7 amino acid residues in length, or can have a length of at least 25 amino acid residues; alternatively, the peptide has a length of at least 50 amino acid residues, or a length of at least 75 amino acid residues. In certain embodiments, the amino acid sequence in the peptide in the composition is present in at least two iterations. Any of the above can further comprise a pharmaceutically acceptable carrier, salt or buffer.

In general, the peptide is capable of binding to class II MHC protein HLA-DR2. Accordingly, the composition in certain embodiments is present in a unit dose effective for treatment of a subject for an autoimmune condition. For example, the autoimmune condition is a cell mediated disease, for example, the autoimmune condition is a demyelinating condition, for example, is multiple sclerosis (MS), diabetes, or Hashimoto's thyroiditis. The cell mediated autoimmune condition is, for example, mediated by a T cell or a natural killer (NK) cell. Alternatively, the autoimmune condition is an antibody mediated disease, for example, systemic lupus erythematosus (SLE) or myasthenia gravis. Any of the compositions herein can further comprise a pharmaceutically acceptable carrier, salt or buffer. In certain embodiments, the peptide is orally available.

Another featured embodiment of the invention herein is an isolated peptide composition having an amino acid sequence capable of inhibiting an immune response in a subject to an autoantigen associated with multiple sclerosis, wherein a position in the amino acid sequence of the peptide that corresponds to an antigen binding pocket in a peptide binding groove of an MHC class II protein HLA-DR2 is identified as a particular amino acid, and the amino acid residue in the position of the sequence that corresponds to the P1 pocket in peptide binding groove of the MHC class II protein is selected from the group consisting of a tyrosine, a valine, a tryptophan and a phenylalanine; and the amino acid residue in the position that corresponds to the P4 pocket is a large hydrophobic amino acid selected from the group consisting of a methionine, a phenylalanine, a tyrosine, and a tryptophan; and wherein the amino acid residue located eight residues beyond, i.e., the P9 pocket, which is eight residues beyond the first amino acid position of the sequence that corresponds to the P1 pocket in the MHC class II peptide binding groove, is a small neutral amino acid; and wherein any residue except that in the P1, P4 and P5 is replaced by a small neutral amino acid. For example, the small neutral amino acid is selected from the group consisting of alanine, serine and glycine residues. The amino acid sequence of the peptide can further comprise at least one proline residue located at a position selected from the terminal and penultimate amino acid residues.

Also featured is an isolated peptide composition that binds to a MHC class II protein HLA-DR2 and stimulates proliferation of Th2 secreting T cells. The Th2 secreting T cells secrete at least one cytokine selected from the group consisting of IL-4, IL-10 and IL-13.

An embodiment of the invention provides a method for treating a demyelinating condition in a subject, the method comprising administering to the subject a composition having at least one peptide selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), APEAKKVEAFKAAAAPA (SEQ ID NO: 17), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26).

An embodiment of the invention provides a method of preventing or reducing symptoms of a demyelinating condition in a subject, the method comprising administering to the subject a composition having at least one peptide selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), APEAKKVEAFKAAAAPA (SEQ ID NO: 17), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26). Accordingly in a related embodiment of this method, administering further includes preventing development of symptoms in the subject. In some embodiments of the method, the subject is asymptomatic. In some embodiments of the method, the subject has an HLA-DR2 haplotype. For example, the haplotype is selected from the group consisting of DRA*0101, DRB1*1501 and alleles related to DRB1*1501, DRB1*1601 and alleles related to DRB1*1601, DQA1*0102, and DQB1*0602. “Related alleles” refers to haplotypes that are substantially identical, such that a minor change in gene sequence or in “neutral” replacements of one or a few amino acids does not affect the binding characteristics of the encoded class II MHC protein DR2. As used herein, the term, “replacement” refers to a different amino acid at a particular residue location in the parent amino acid sequence, i.e., an amino acid carrying a different side chain at the a position in the carbon chain is located at the same residue compared to the parent peptide amino acid sequence. The replacement may be may another naturally occurring amino acid, or by a non-naturally occurring amino acid.

In embodiments of the invention related to any of these methods, prior to administering, the method can further comprise testing T cells from the subject for reactivity to a dominant autoimmune epitope for MS. For example, the epitope is a myelin basic protein (MBP) peptide, for example, the epitope is MBP 85-99. In embodiments related to any of these methods, the subject is a mammal, for example, a rodent, for example, the rodent is a mouse with experimental allergic encephalomyelitis, or the rodent is a humanized mouse. The subject mammal in various embodiments of the method is a human, for example, the subject is a patient with MS. Accordingly, in related embodiments, treating the subject or reducing the symptom is decreasing severity or decreasing frequency of recurrences of symptoms.

A variety of different routes of administration are envisioned for the compositions and methods herein, for example, the peptide is administered as a bolus injection, or the peptide is orally administered. Injection can be intravenous (i.v.), subcutaneous (s.c.), intramuscular (i.m.), and intraperitoneal (i.p.). Alternatively to administering as a bolus injection, the peptide in certain embodiments is administered as an intravenous infusion.

Other embodiments of the invention further comprise, after administering the composition, analyzing a physiological parameter of the demyelinating condition. For example, the physiological parameter is testing T cells from the subject for reactivity to a peptide of myelin basic protein, for example, the peptide is MBP 85-99.

In related embodiments, the method can further involve administering an additional therapeutic agent. The additional therapeutic agent is selected from an antibody, an enzyme inhibitor, an antibacterial agent, an antiviral agent, a steroid, a nonsteroidal anti-inflammatory agent, an antimetabolite, a cytokine, a cytokine blocking agent, an adhesion molecule blocking agent, a soluble cytokine receptor, and a random linear amino acid copolymer composition. For example, the cytokine is an interferon. For example, the copolymer is selected from the group of YEAK (Copaxone®), YAK, FYAK, VWAK and VFAK. The copolymer is YEAK, or is YAK. Alternatively, the copolymer is at least one of FYAK, VWAK and VFAK. Further, the immune suppressant is, for example, a drug or a protein; in various embodiments, the drug is at least one of rapamycin and FK506, and the protein is OKT3.

Another embodiment of the invention herein is a method of inhibiting secretion of an interleukin or a cytokine by an HLA-DR-2-restricted T cell, by contacting the T cell with at least one composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41).

In a related embodiment of this method the interleukin or cytokine is interleukin-2 (IL-2) or gamma interferon (IFN-γ). Thus in general, the interleukin is a non-Th2 interleukin. A related method further includes observing inhibition of secretion that is greater than an inhibition observed in cells contacted with a random amino acid polymer. In another related embodiment this method includes observing stimulation of a Th2 interleukin. Accordingly, the Th2 interleukin comprises interleukin-4 (IL-4) or interleukin-10 (IL-10).

Also provided herein is a method of inhibiting expansion of an antigen-specific T cell comprising contacting the T cell with at least one composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41). In a related embodiment this method further includes observing that the contacted T cell fails to increase proliferating. In a related embodiment, the T cell is specific to a demyelinating condition, for example, the autoimmune T cell is specific to multiple sclerosis. In certain embodiments, the T cell is in a subject.

Also provided herein is a method of ameliorating development of symptoms of a demyelinating condition in a subject, comprising administering to the subject a composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41). In certain embodiments, the subject is asymptomatic. Accordingly in a related embodiment, the subject carries an HLA-DR2 haplotype. In certain embodiments, the haplotype comprises at least one allele selected from the group of: DRA*0101, DRB1*1501, DQA1*0102, and DQB1*0602. Further in certain embodiments, the subject contains a greater number of T cells that react specifically with a myelin specific autoantigen than a member of the general population. In certain embodiments the autoantigen is selected from MBP 85-99 and PLP 139-151.

The invention in another embodiment provides a method of manufacture of a medicament for treatment of a demyelinating condition, comprising formulating a peptide having a sequence of amino acids according to any of the above compositions, and administering to a subject. The invention in another embodiment provides a use of a peptide having a sequence of amino acids according to any of the above compositions, for treating a subject having a demyelinating condition.

Further, the invention in another embodiment provides a kit for treating a subject having a demyelinating condition, the kit including a peptide having a sequence of amino acids according to any of the above compositions, and a container. The kit in a related embodiment further includes instructions for use. The kit in a related embodiment can further include the peptide in a unit dose.

### BRIEF DESCRIPTION OF THE DRAWINGS

FIG. 1 is a set of line graphs and bar graphs showing that novel synthetic peptides inhibit binding of MBP 85-99 to HLA-DR2 (DRB1*1501) and inhibit antigen presentation.

FIG. 1A shows data from recombinant HLA-DR2 molecules that were incubated with 0.13 μM of biotinylated MBP 85-99 alone, or the biotinylated MBP 85-99 and various concentrations of unlabeled MBP 85-99 or peptides J2, J3, J4 or J5 (1-100 μM; SEQ ID NOs: 2, 3, 4 or 5, respectively). The reaction mixtures were transferred to plates coated with LB3.1 Ab specific to HLA DR2 molecules. Following addition of streptavidin-conjugated alkaline phosphatase, absorbance was measured at 450 nm by using a microtiter plate reader. Percent inhibition was calculated as described in the Materials and Methods.

FIG. 1B shows data obtained using irradiated MGAR cells as antigen presenting cells (APC), co-incubated with MBP 85-99 (12.5 μM) and various concentrations of the synthetic peptides or Cop1 for 2 h. Then MBP 85-99-specific hybridoma T cells were added, and the mixtures were incubated for 24 h. Culture supernatants were used to test the proliferative response of IL-2-dependent CTLL and the response was measured as counts per minute (CPM) after pulsing with ³H-thymidine.

FIG. 2 is a set of line graphs showing that the synthetic peptides are immunogenic. FIG. 2A shows data on splenocyte proliferative response obtained using humanized mice that were immunized with MBP 85-99 (solid diamond symbols), or with each of peptides J2, J3, and J5 (SEQ ID NOs: 2, 3 and 5, respectively, shown as open symbols in each respective panel). After 10 days, splenocytes were re-stimulated for three rounds with their corresponding peptides in the presence of irradiated antigen presenting cells (APCs). Two weeks later, viable cells were stimulated with corresponding peptides for two days. Cells were contacted for sixteen hours with ³[H]-thymidine, and the proliferative response was measured as CPM. A peptide indicated Nase, shown in solid black symbols, having residues 101-120, EALVRQGLAKVAYVYKPNNT (SEQ ID NO: 27) of a neuraminidase was used as a negative control in T cell proliferation experiments.

FIG. 2B shows similar data obtained using SJL/J mice (in which I-A^{S} is the only expressed class II major histocampatilbity complex protein) immunized with PLP 139-151 or with each of peptides J2, J3, and J5.

FIG. 3 is a bar graph and a line graph showing that each of peptides J2, J3 and J5 (SEQ ID NOs: 2, 3 and 5, respectively) do not stimulate MBP 85-99- or PLP 139-151-specific T cells.

FIG. 3A shows data obtained using MBP 85-99-specific transgenic (tg) and endogenous (non-tg) cells from humanized mice that were cultured with MBP 85-99 or with each of peptides J2, J3 and J5 (SEQ ID NOs: 2, 3 and 5, respectively) and were then sorted by flow cytometry. Cells were cultured with MBP 85-99 or peptides J2, J3 or J5, and their proliferative responses were measured as counts per minute (CPM) after pulsing with ³[H]-thymidine.

FIG. 3B shows data from PLP 139-151-specific T cells that were incubated with PLP 139-151 or the novel peptides. The proliferative responses were measured as CPM after pulsing with ³[H] thymidine.

FIG. 4 is a set of bar graphs showing that each of peptides J2, J3 and J5 (SEQ ID NOs: 2, 3 and 5, respectively) induce Th2 cytokine secretion. SJL/J mice were immunized with either PLP 139-151 or each of the peptides J2, J3, and J5 (SEQ ID NOs: 2, 3 and 5, respectively). After ten days, single cell suspensions derived from lymph nodes and spleens were re-stimulated with the corresponding peptides in the presence of antigen presenting cells for two days. Culture supernatants were analyzed for amount of secretion of IL-2, IL-4, IL-10 and IFN-γ by ELISA.

FIG. 5 is a set of line graphs showing that the synthetic peptides herein reduce severity of symptoms of EAE. EAE was induced in humanized or in SJL/J mice by immunizing with MBP 85-99 or PLP 139-151, respectively, and mice were further treated with each of novel peptides J2, J3, and J5 (SEQ ID NOs: 2, 3 and 5, respectively) or Cop1; control mice identified by solid diamond symbols were not further treated with peptides or Cop1. Appearance of clinical signs of EAE was monitored daily, and the disease severity was scored as described in the Materials and Methods.

FIG. 6 is a line graph showing suppression of EAE upon adoptive transfer of peptide-specific T cell lines. Peptide-specific T cells (5×10⁶) from lines established after immunization with J2, J3 or J5 (250 μg), or controls from untreated mice were transferred into naïve SJL/J mice, and mice were immunized subcutaneously a day later with 50 μg of PLP 139-151 in complete Freund's adjuvant (CFA) to induce EAE. Clinical symptoms were monitored and scored daily.

FIG. 7 is a read-out of a cell sorting procedure, showing that each of peptides J2, J3, and J5 (SEQ ID NOs: 2, 3 and 5, respectively) suppress antigen-specific CD4⁺ T cell response. SJL/J mice were immunized with PLP 139-151 in CFA or were co-immunized with this peptide and each of J2, J3 and J5: (panel A) PLP 139-151 alone; PLP 139-151 co-immunized with (panel B) J5 (SEQ ID NO: 5), (panel C) J2 (SEQ ID NO: 2), or (panel D) J3 (SEQ ID NO: 3). After 10 days, lymphocytes were cultured with PLP 139-151. Four days later, after gating with anti-CD4, anti-CD25, and 7-amino-actinomycin D (7-AAD), live cells were incubated with I-A^{s} tetramers with PLP 139-151 (left) or with control peptide TMEV 70-86 (right) for 3 hrs. The I-A^{s}/tetramer-stained cells were determined in live CD4⁺ T cell subset after eliminating dead cells (7-AAD positive).

### DESCRIPTION OF SPECIFIC EMBODIMENTS

It is shown herein that by improving the binding capacity of amino acid copolymers or synthetic peptide analogues (see also Fridkis-Hareli, M. et al. 2001 Hum Immunol 62:753; Illés, Z. et al. 2004. PNAS 101:11749; Stern, J. N. et al. 2004. PNAS 101:11743. Illés, Z. et al. 2004. PNAS 101:11749 and Stern, J. N. et al. 2004. PNAS 101:11743 are hereby incorporated herein in their entireties), blocking of antigen presentation is effectively enhanced. Thus T cell recognition of natural ligands is more effectively inhibited and/or additional immunosuppressive functions of the copolymers are mimicked by peptides. Improved peptide inhibitors were designed herein based on the motif of binding of MBP85-99 to HLA-DR2. Three novel peptide 15mers provided herein (J2, J3, and J5, identified as SEQ ID NOs: 2, 3 and 5, respectively) are shown to have the following properties: each of the peptides was found to compete with MBP85-99 for binding to HLA-DR2, inhibit IL-2 secretion by MBP85-99-specific T cell clones, induce production of Th2 cytokines by splenocytes, and not cross-react with natural ligand-specific T cells. They also were found to suppress EAE in two different murine disease models.

Previously, peptides have been described whose design was based on a binding motif of Cop1 to HLA-DR2. (See U.S. patent application Ser. No. 10/438,538 published as 20040006022A1 Jan. 8, 2004; and U.S. Pat. No. 6,930,168 issued Aug. 16, 2005). Precise constituents of random mixtures are not as exactly reproducible from batch to batch as peptides having a defined sequence. Cop1 reduces the frequency of relapse in MS by only about 30%. Cop1 is a random copolymer of four amino acids, Y, E, A and K, in the molar ratio 1.0, 1.4, 4.2, and 3.4, and is made in solution by polymerization of N-carboxy amino acid anhydrides (Teitelbaum, D. et al. 1971. Eur J Immunol 1:242; Teitelbaum, D. et al. 1973. Eur J Immunol 3:273). The length of the copolymer produced varies from 50 to 80 amino acids. Copolymers of 50 amino acids in length or larger are biologically active. Considerable reduction in efficacy occurs with copolymers of 35 amino acids in length, and random peptides of 15 amino acids (the size of the groove in HLA-DR2 to which peptides and copolymers bind) are essentially inactive (Fridkis-Hareli, M. et al. 2002. J Clin Invest 109:1635). Based on the data presented here, a size of 50 or larger may be required in order to generate, within the random copolymers, a specific sequence or sequences in sufficient quantity to produce the desired effect.

Peptides of 15 amino acids in length were previously designed based on a binding motif of Copolymer 1 to HLA-DR2. Approximately 120 peptides were synthesized and assayed by their ability to compete with MBP85-99 for binding to HLA-DR2, and for inhibition of proliferation of two T cell hybridomas that carry the TCR derived from two different MS patients (Fridkis-Hareli, M. et al. 2001 Hum Immunol 62:753). Among the peptides synthesized, two peptides (#101 and #102, Fridkis-Hareli, M. et al. 2001 Hum Immunol 62:753, identified as J1 and J11 herein were unusually active in both assays. These peptides had minimum similarity to MBP85-99. However all retained K at position P5, the dominant T cell stimulatory residue (Wucherpfennig, K. W. et al. 1994. J Exp Med 179:279; Smith, K. J. et al. 1998. J Exp Med 188:1511), and had a large hydrophobic amino acid, Y, at both putative anchor position residues P1 and P4.

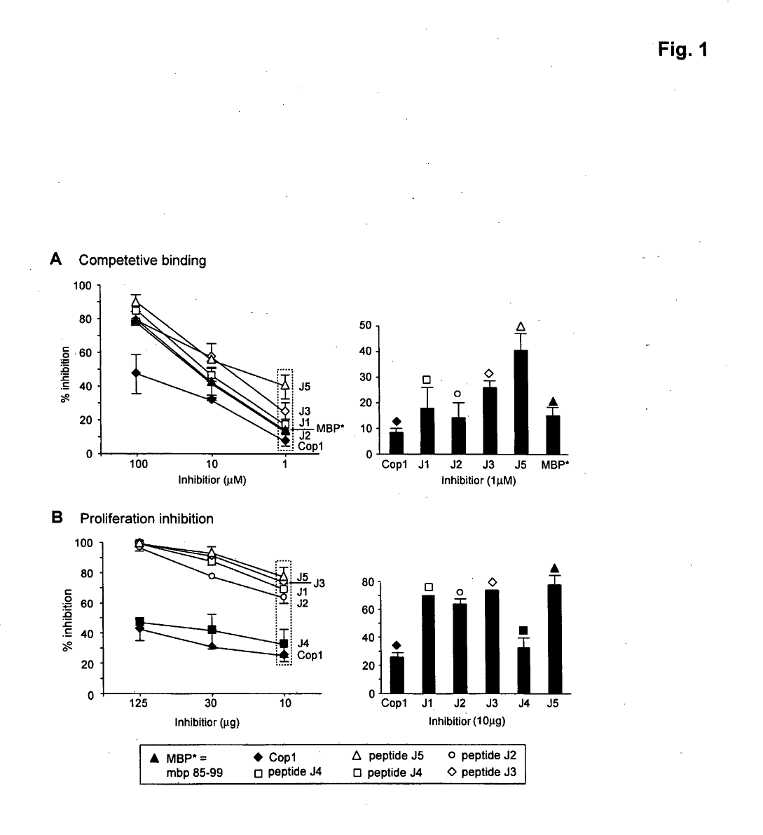

To improve the binding affinity of peptides for HLA-DR2, peptide sequences are designed using knowledge of how peptides bind to this protein (Wucherpfennig, K. W. et al. 1994. J Exp Med 179:279; Smith, K. J. et al. 1998. J Exp Med 188:1511) as well as their ability to inhibit encephalitogenic T cell responses in vitro, and more importantly, these peptides are tested in in vivo assays of amelioration of EAE in mice. The following features are found in design of the peptides. Peptides J1 and J11 (#100 and #101 in Fridkis-Hareli, M. et al. 2001 Hum Immunol 62:753) have Y at P4, which is well accommodated in the large hydrophobic P4 pocket of HLA-DR2 (FIG. 1, Table 1). Further, these peptides have Y at the putative P1 position, an amino acid which is too large for the P1 pocket. This small pocket is formed in part by β86V (rather than β86G) of HLA-DR2, and Y can be forced into it only at high concentrations. Thus, amino acid V, which is found at P1 in MBP85-99, and F, which may just fill the P1 pocket, are herein systematically replaced at this position. Two peptides provided herein, J4 and J6, are designed to contain E at the putative P1 position. The negative charge of E is herein found to preclude binding to HLA-DR2, and these peptides henceforth serve as controls. Finally, amino acid P found as P87 in MBP85-99 was added at this position in some peptides, either as the penultimate 14^{th} residue, or at the very beginning of the peptide, to prevent degradation by amino- and carboxy-peptidases found in serum. In some of the peptides provided herein, the putative P4 position was supplied with F, because the very large hydrophobic P4 pocket of HLA-DR2 can accommodate F, Y or even W (Wucherpfennig, K. W. et al. 1994. J Exp Med 179:279).

Most of the peptides provided herein were found to inhibit binding of biotinylated MBP85-99 to some extent, as did Cop1 (FIG. 1, Table 1). Further, several peptides were found that inhibited binding much better than the others, i.e., inhibited binding greater than 25% at the lowest concentration tested. In fact, these peptides were found to be far more effective in competing with biotinylated MBP than either MBP85-99 itself, or than Cop1. These same peptides were also the best at inhibiting proliferation of the T cell hybridomas (FIG. 1).

The peptides were evaluated in vivo. Peptide J2 (the proline replaced derivative of J1) and both J3 and J5 (also with proline replacements, and with F or V at P1, respectively) were examined.

Two different mouse strains were employed. SJL/J mice bear I-A^{s} as the class II MHC protein, while in the humanized mouse strain, all murine class II genes have been genetically knocked out and replaced by DRA and DRB1*1501 that encode the HLA-DR2 molecule. In addition, this mouse bears as a transgene the TCR from an MS patient (the same TCR that is present in the 8073 T cell hybridoma). These mice were immunized with peptide MBP85-99 (humanized mouse) or PLP139-151 (SJL/J mouse). Splenocyte lines were established by stimulation with these same peptides, or with J2, J3 and J5 peptides.

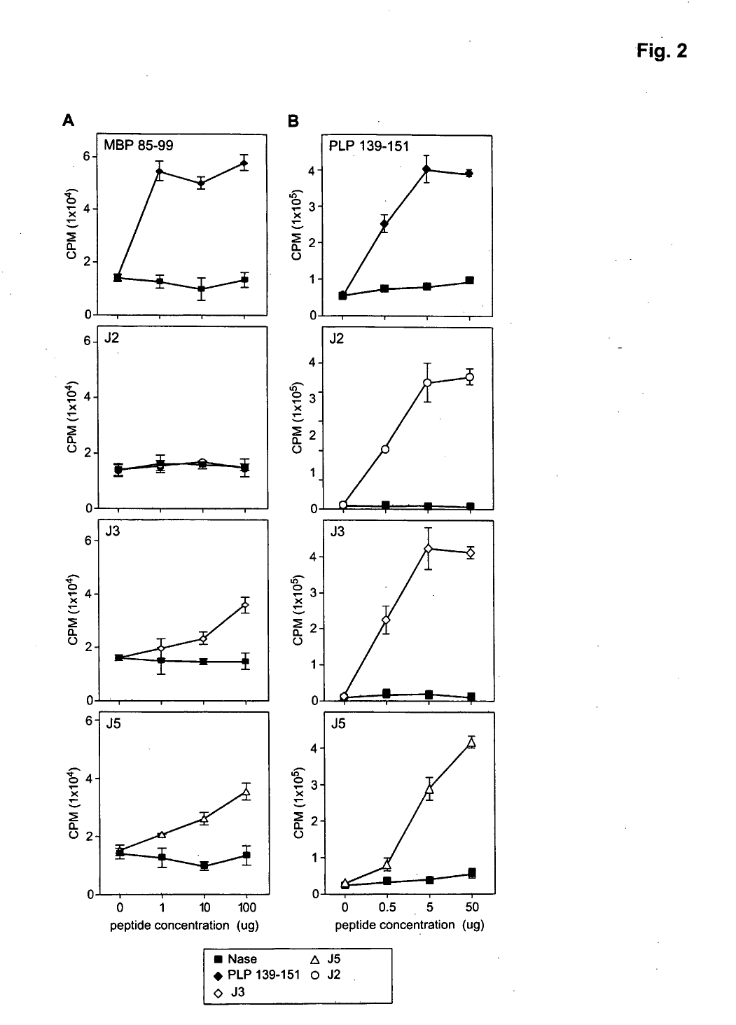

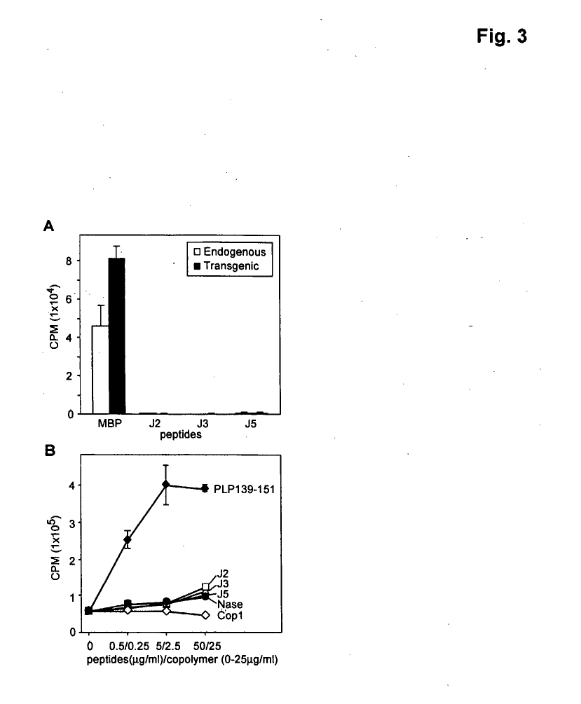

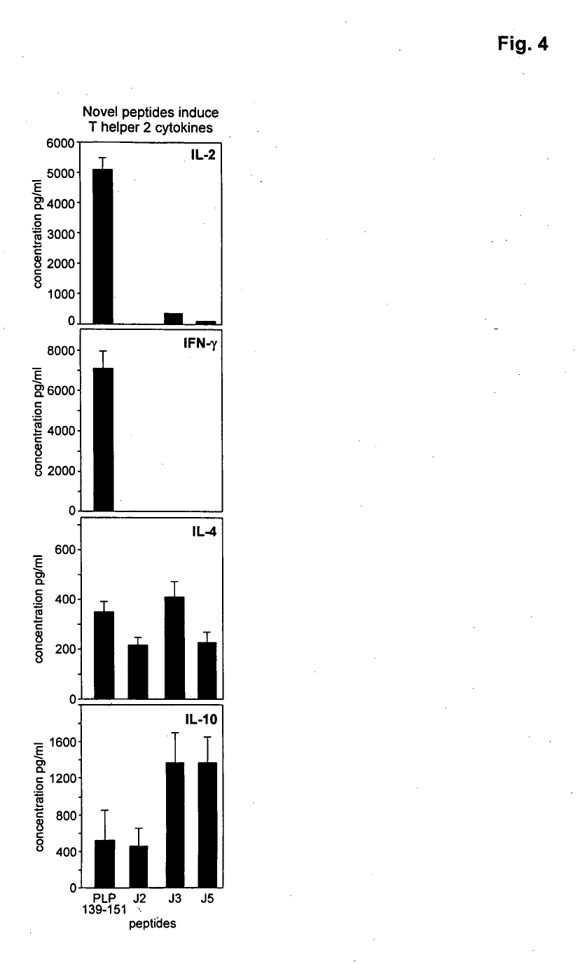

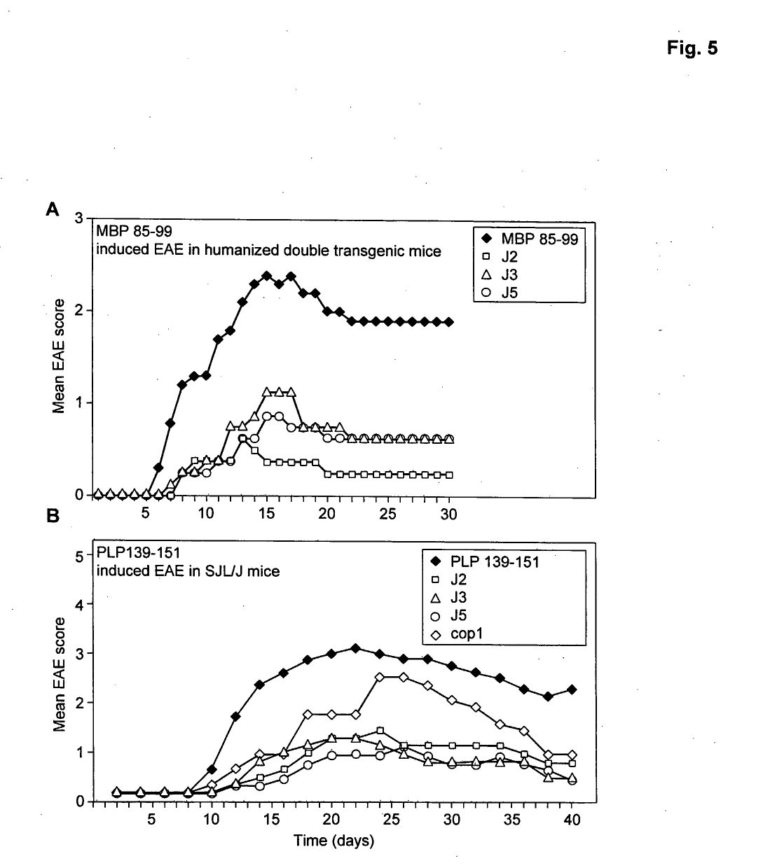

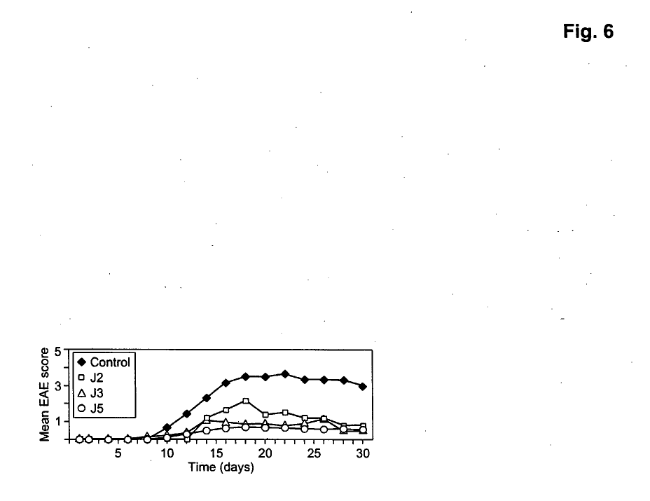

Proliferative responses showed that these lines were specific for each of the immunogens (except in the case of the line established from the humanized mouse with the J2 peptide in which case proliferation was very weak; FIG. 2). Notably, the lines that responded to MBP85-99 or PLP139-151 did not cross-react with any of the peptides J2, J3 and J5 or with Cop1 (FIG. 3). The peptides were also remarkably effective in the amelioration of EAE in both mouse models in comparison to Cop1 (FIG. 5). These data show that the peptides herein prevent the expansion of cognate ligands that induce EAE based on the following evidence. The peptide 15mers tested (J2, J3 and J5) effectively competed with MBP85-99 in binding assays in vitro (FIG. 1, Table 1). With class II MHC system bearing I-A^{s} haplotype that is unrelated, the peptides herein induced a dominant immune response comparable to that of natural ligand, PLP139-151 in SJL/J mice (FIG. 4). Surprisingly in SJL/J mice, the synthetic peptides, specifically J2 and J5, prevented expansion of PLP139-151-specific CD4 cells (FIG. 6), showing that the novel peptides mediate their effects in individuals with diverse MHC haplotypes.

Previously, an altered MBP 85-99 was designed by introducing the replacement peptides having E83A, N84K, V89L (P1) and F91A compared to the parent amino acid sequence for MBP85-99 (Kappos, L. et al. 2000. Nat Med 6:1176). This APL was shown to be effective in binding to HLA-DR2 molecules, and also to be proved therapeutically beneficial in an animal model of MS (Karin, N. et al. 1994. J Exp Med 180:2227). However, the APL exacerbated the clinical signs of MS, an effect attributed to cross-reactivity with MBP 85-99 (Bielekova, B. et al. 2000. Nat Med 6:1167).

Induction of EAE is mediated by Th1 cells, and IFN-γ is an important mediator of encephalomyelitis (Nagelkerken, L. 1998. Braz J Med Biol Res 31:551; Druet, P. et al. 1995. Clin Exp Immunol 101 Suppl 1:9). Further, Th2 cytokines (IL-4 and IL-10) have been shown to posses anti-inflammatory properties, neutralizing the effects of IFN-γ. Mice transgenic for IL-10 are highly resistant to myelin oligodendrocyte glycoprotein (MOG)35-55-induced EAE (Bettelli, E. et al. 1998. J Immunol 161:3299; Bettelli, E. et al. 2003. J Autoimmun 20:265). Since these synthetic peptides herein induced dominant Th2 cytokines, they might have contributed to the reduction of disease severity in MBP85-99-induced EAE models. Induction of cell death of antigen-specific T cells was not a possible explanation, since no non-specific cell death was seen in cultures treated with the synthetic peptides herein alone.

Thus, 15mer peptides of defined sequence have been synthesized that can be used in lieu of random amino acid copolymers in the amelioration of EAE. Three such copolymers have been described, viz. Copolymer 1 [YEAK, poly(Y,E,A,K)n], FYAK [poly(F,Y,A,K)n], and VWAK [poly(V,W,A,K)n] (Illés, Z. et al. 2004. PNAS 101:11749; Stern, J. N. et al. 2004. PNAS 101:11743). The present peptides as well as the three copolymers appear to share the ability to induce the immunosuppressive cytokines IL-4 and IL-10. Among these agents, FYAK and peptides J3 and J5 are the most effective in inducing these cytokines. The lack of cross-reactivity with cell lines specific for either MBP85-99 or PLP139-151 indicate that the new synthetic peptides provided herein may be suitable for clinical trials in human subjects with MS.

Unless the context otherwise requires, as used in this description and in the following claims, the terms below shall have the meanings as set forth:

The term “autoimmune condition” or “autoimmune disease” means a disease state caused by an inappropriate immune response that is directed to a self-encoded entity which is known as an autoantigen. The copolymer compounds provided herein can be used to treat symptoms of an autoimmune disease, a class of disorder which includes Hashimoto's thyroiditis; idiopathic myxedema, a severe hypothyroidism; multiple sclerosis, a demyelinating disease marked by patches or hardened tissue in the brain or the spinal cord; myasthenia gravis which is a disease having progressive weakness of muscles caused by autoimmune attack on acetylcholine receptors at neuromuscular junctions; Guillain-Barre syndrome, a polyneuritis; systemic lupus erythematosis; uveitis; autoimmune oophoritis; chronic immune thrombocytopenic purpura; colitis; diabetes; Grave's disease, which is a form of hypothyroidism; psoriasis; pemphigus vulgaris; and rheumatoid arthritis (RA). Autoimmune diseases are further considered cell mediated or antibody mediated. Cell mediated autoimmune diseases arise from activities of lymphocytes such as T cells and natural killer cells, while antibody mediated diseases are caused by attack of antibodies produced by B cells and secreted into the circulatory system. Examples of cell mediated autoimmune conditions or diseases are diabetes, multiple sclerosis, and Hashimoto's thyroiditis. Examples of antibody mediated conditions or diseases are systemic lupus erythematosus and myasthenia gravis.

The term “demyelinating condition” includes a disease state in which a portion of the myelin sheath, consisting of plasma membrane wrapped around the elongated portion of the nerve cell, is removed by degradation. A demyelinating condition can arise post-vaccination, post-anti TNF treatment, post-viral infection, and in MS.

The term “derivative” of an amino acid means a chemically related form of that amino acid having an additional substituent, for example, N-carboxyanhydride group, a γ-benzyl group, an ε,N-trifluoroacetyl group, a methyl or any other alkyl group, an acetyl or any other carboxyalkyl alkyoxy group, a formyl, an amide, or a halide group attached to an atom of the amino acid.

The term “analog” means a chemically related form of that amino acid having a different configuration, for example, an isomer, or an enantiomer such as an amino acid in D-configuration rather than an L-configuration, or an organic molecule with the approximate size, charge, and shape of the amino acid, or an amino acid with modification to the atoms that are involved in the peptide bond, so that the copolymer having the analog residue is more protease resistant than an otherwise similar copolymer lacking such analog, whether the analog is interior or is located at a terminus of the copolymer, compared to the copolymer without the analog.

The phrases “amino acid” and “amino acid peptide” or “peptide” can include one or more components which are amino acid derivatives and/or amino acid analogs as defined herein, the derivative or analog comprising part or the entirety of the residues for any one or more of the 20 naturally occurring amino acids indicated by that composition. For example, in an amino acid peptide composition having one or more tyrosine residues, a portion of one or more of those residues can be replaced by homotyrosine. Further, an amino acid peptide having one or more non-peptide or peptidomimetic bonds between two adjacent residues, is included within this definition.

The term “hydrophobic” amino acid means aliphatic amino acids alanine (A, or ala), glycine (G, or gly), isoleucine (I, or ile), leucine (L, or leu), methionine (M, or met), proline (P, or pro), and valine (V, or val), the terms in parentheses being the one letter and three letter standard code abbreviations for each amino acid, and aromatic amino acids tryptophan (W, or trp), phenylalanine (F, or phe), and tyrosine (Y, or tyr). These amino acids confer hydrophobicity as a function of the length of aliphatic and size of aromatic side chains, when found as residues within a copolymer or other polypeptide.

The term “charged” amino acid means amino acids aspartic acid (D or asp), glutamic acid (E or glu), arginine (R or arg) and lysine (K or lys), which confer a positive (lys, and arg) or negative (asp, glu) charge at physiological values of pH on an aqueous solution of a peptide or other amino acid composition containing one or more residues of these amino acids. Histidine (H or his) is hydrophobic at pH 7, and charged at pH 6.

The term “anergy” means unresponsiveness of the immune system of a subject to an antigen.

The term “subject” as used herein indicates a mammal, including a human. The term “patient” is generally used to indicate a human subject suffering from a disease or health condition.

The term “heterologous cell” means a cell for production of an MHC protein which is unrelated to a cell of a subject, e.g., the heterologous cell is not a cell of a mammal. The heterologous cell for example can be from a cold blooded animal, for example, from an invertebrate; the heterologous cell is an insect cell, or a cell of a microorganism such as a yeast cell.

The term “surfaces of Class II MHC HLA-DR-2 protein” includes the portions of the protein molecule in its three-dimensional configuration which are in contact with its external environment, including those features of the protein that interact with aqueous solvent and are capable of binding to other cell components such as nucleic acids, other proteins, and peptides.

The terms “P1 pocket” and “P4 pocket” include three dimensional polymorphic regions on the peptide binding surface of the Class II MHC protein molecule that accommodate amino acid residue side chains from a peptide that is bound to the Class II MHC protein (Fridkis-Hareli, M. et al. 1998. J. Immunol. 160:4386-4397; Fridkis-Hareli, M. et. al. 2000. Human Immunol. 61:640; Fridkis-Hareli, M. et al. 2001. Human Immunol. 62:753-763), including a bound naturally occurring antigen or epitope, and a bound synthetic peptide or copolymer.

The terms “P−1 position” and “P5 position” refer to amino acid residues on the Class II MHC protein molecule peptide complex which directly contact the T-cell receptor (Fridkis-Hareli, M. et. al. 2000. Human Immunol. 61:640; Fridkis-Hareli, M. et al. 2001. Human Immunol. 62:753-763). The P−1 position refers to the amino acid which precedes the amino acid residue of the peptide that occupies the P1 pocket. The P5 position refers to the amino acid residue that follows the amino acid residue that occupies the P4 pocket.

The term “antigen binding groove” refers to a three dimensional antigen interactive site on the surface of the Class II MHC protein molecule (Stem, L. J. et. al., 1994. Nature 368:215) that is formed by surfaces of both the α and β subunits of the Class II MHC protein molecule.

The term “pharmaceutically acceptable carrier” includes any and all solvents, dispersion media, coatings, antimicrobials such as antibacterial and antifungal agents, isotonic and absorption delaying agents and the like that are physiologically compatible. Preferably, the carrier is suitable for intravenous, intramuscular, oral, intraperitoneal, transdermal, or subcutaneous administration, and the active compound can be coated in a material to protect it from inactivation by the action of acids or other adverse natural conditions.

An autoimmune disease results when a host's immune response fails to distinguish foreign antigens from self molecules (autoantigens) thereby eliciting an aberrant immune response. The immune response towards self molecules in an autoimmune disease results in a deviation from the normal state of self-tolerance, which involves the destruction of T cells and B cells capable of reacting against autoantigens, which has been prevented by events that occur in the development of the immune system early in life. The cell surface proteins that play a central role in regulation of immune responses through their ability to bind and present processed peptides to T cells are the major histocompatibility complex (MHC) molecules (Rothbard, J. B. et al., 1991. Annu. Rev. Immunol. 9:527).

In addition to MS, other demyelinating conditions have been found to occur, for example, post-viral infection, post-vaccination, post-encephalomyelitis (Wucherpfennig K. W. et al. 1991. Immunol. Today 12:277-282) and following administration of certain anti-TNF agents (FDA Talk Paper, Food and Drug Administration Public Health Service, Rockville, Md., http://www.fda.gov/bbs/topics/ANSWERS/ANSDO954.html).

Peptides of Amino Acids as Therapeutic Agents for Autoimmune Diseases

Methods of the invention include use of a class of agents that can bind to Class II MHC proteins encoded by particular alleles. Such an agent can bind to a particular Class II MHC protein, and thus inhibit and/or prevent the binding of an autoantigen involved in an autoimmune disease, or upon binding can induce anergy, so that there is no response of the immune system to the autoantigen.

A number of therapeutic agents have been developed to treat autoimmune diseases. For example, agents have been developed that can, by inhibiting a cyclooxygenase, prevent formation of low molecular weight inflammatory compounds. Also, agents are available that can function by inhibiting a protein mediator of inflammation, by sequestering the inflammatory protein tumor necrosis factor (TNF) with an anti-TNF specific monoclonal antibody fragment, or with a soluble form of the TNF receptor. Finally, agents are available that target and inhibit the function of a protein on the surface of a T cell (the CD4 receptor or the cell adhesion receptor ICAM-1) thereby preventing a productive interaction with an antigen presenting cell (APC). However, compositions which are natural folded proteins as therapeutic agents can incur problems in production, formulation, storage, and delivery. Further, natural proteins can be contaminated with pathogenic agents such as viruses and prions.

An additional target for inhibition of an autoimmune response is the set of lymphocyte surface proteins represented by the MHC molecules. Specifically, these proteins are encoded by the Class II MHC genes designated as HLA (human leukocyte antigen) -DR, -DQ and -DP. Each of the MHC genes is found in a large number of alternative or allelic forms within a mammalian population. The genomes of subjects affected with certain autoimmune diseases, for example, MS and rheumatoid arthritis (RA), are more likely to carry one or more characteristic Class II MHC alleles, to which that disease is linked.

A potential source of agents for treatment of MS and other demyelinating conditions is to identify peptides that bind selectively in vitro to a purified Class II MHC allele protein molecule, particularly to a protein which is a product of an Class II MHC allele associated with demyelinating conditions. In addition, the agent should bind to that protein as it occurs on the surfaces of antigen presenting cells in vivo, and thereby block, anergize, or inactivate the class of T cells that are responsible for the demyelinating condition, such as MS.

The Class II MHC protein consists of two approximately equal-sized subunits, α and β, which are transmembrane proteins. A peptide-binding cleft, which is formed by protein features of both α and β subunits, is the site of presentation of the antigen to T cells. There are at least three types of Class II MHC molecules: HLA-DR, -DQ, and -DP, and there are numerous alleles of each type. The Class II MHC molecules are expressed predominantly on the surfaces of B lymphocytes and antigen presenting cells such as macrophages and dendritic cells (Mengle-Gaw, L., The Major Histocompatibility Complex (MHC), in the Encyclopedia of Molecular Biology, Oxford: Blackwell Science Ltd., 1994, pp. 602-606).

An embodiment of the invention includes a novel method for treating autoimmune diseases, by targeting Class II MHC molecules with a class of compounds identified as peptides that include different amino acids in a specific sequence or arrangement that is uniform throughout the formulation of the composition.

A peptide of the invention can be synthesized using fmoc- or tboc-protected initiating amino acid analogs, or the like, which are immobilized on a resin in an automated peptide synthesis apparatus for further polymerization (solid state synthesis). The amino acids are polymerized in a sequence that can be controlled to provide a peptide of amino acid sequence with optimal binding characteristics.

Synthesis procedures can include providing a solution of each of the chosen amino acids in an activated form, for example, activated as an N-carboxy anhydride, in the appropriate derivatized amino acid precursors (derivatized to protect certain functional groups, such as the ε amino group of L-lysine, for example the precursor ε,N-trifluoroacetyl-L-lysine). Peptide synthesis services can be obtained commercially, for example, at Chiron Technologies, Clayton, Australia, the Harvard Medical School Biopolymer Laboratory, Boston, Mass., and at Advanced ChemTech, Inc., Louisville, Ky.

Examples of such resin supports for peptide synthesis include a Merrifield resin, chloromethylated polystyrene with 1% DVB cross-links; an fmoc amino acid Wang resin, 4-benzyloxybenzyl alcohol, the resins being pre-loaded with an amino acid (for example, fmoc-D-trp(boc)-Wang resin). Resins are available in different mesh sizes, for example 100-200 mesh, and high loading or low loading densities of functionalization of the initiating amino acid.

A solution of each of the different derivatized amino acids to be polymerized into the peptide composition of the invention, preferably protected as is conventional in peptide synthesis, is added to sample of beads e.g., fmoc. Reagents for synthesis, for deblocking, and for cleavage of the complete copolymer molecules for removal from the resin are available from manufacturers of the apparatus (Applied Biosystems Peptide Synthesizer, Foster City, Calif., or Advanced ChemTech, Louisville, Ky.); see e.g., M. Bodansky, Principles of Peptide Synthesis, 2nd Ed., Springer-Verlag, 1991, the contents of which are herein incorporated by reference. Additional amino acids or analogs or derivatives of amino acids, can be added to the at least three amino acids selected to comprise the copolymers, to replace all or for a small proportion of one or more of the amino acids in the sequence, to provide, for example, a peptide having increased protease resistance and therefore having enhanced pharmacological properties such as longer in vivo lifetime. Examples of analogs are homotyrosine, or other substituted tyrosine derivatives, and aminobutyric acid, each available as an fmoc derivative from Advanced ChemTech.

The invention in other embodiments provides peptide “analogs” having non-peptide bonds in various proportions to the peptide bonds in the peptide backbone, the presence of non-peptide bonds conferring improved pharmacological properties on the peptide analogs. A peptide analog is synthesized having the same sequence of amino acid precursors as a “parent” peptide, i.e., the parent peptide having peptide bonds and comprising amino acids in a specific sequence, however using an amino acid analog that forms a bond that is not a peptide bond. The peptide analogs are potential therapeutic agents for autoimmune diseases, as they retain the ability of to parent peptide to inhibit or to prevent interaction of an auto-antigenic peptide, e.g., an auto-antigen associated with MS such as MBP 58-99 (SEQ ID) No: 2), to interact with a Class II MHC protein, for example, with Class II MHC DR-2 protein, and to prevent or cure the autoimmune disease such as EAE in mice or MS in humans.

A peptide bond is substituted during synthesis of a peptide analog by a bond selected from the group consisting of: —CH₂NH—(“pseudopeptide bond”), —CH₂S—, —CH₂CH₂—, —CH═CH—(“olefin”, cis or trans), —COCH₂—, —CH(OH)CH₂—, and —CH₂SO—, by methods known in the art and further described in the following references: Coy, D. et al., U.S. Pat. Nos. 4,803,261 and 5,750,646; Spatola, A. F. in “Chemistry and Biochemistry of Amino Acids, Peptides, and Proteins,” B. Weinstein, eds., Marcel Dekker, New York, p. 267 (1983); Soatola, A. F., Vega Data (March 1983), Vol. 1, Issue 3, “Peptide Backbone Modifications” (general review); Spatola, A. F. et al., Life Sci (1986) 33:1243-1249; Hann, M. M., J Chem Soc Perkin Trans I (1982) 307-314; Almquist, R. G. et al., J Med Chem (1980) 23:1392-1398; Jennings-White, C. et al., Tetrahedron Lett (1982) 23:2533; Szelke, M. et al., European Appln. EP 45665 (1982) CA: 97:39405 (1982); Holladay, M. W. et al., Tetrahedron Lett (1983) 24:4401-4404; and Hruby, V. J., Life Sci (1982) 31:189-199; each of which is incorporated herein by reference. Advantages of the peptide analogs, in comparison to the parent peptide having only peptide bonds, include: more economical production, greater chemical stability, enhanced pharmacological properties (half-life, absorption, potency, efficacy, etc.), altered specificity (e.g., a broader or a narrower spectrum of biological activities), and reduced antigenicity. Without being limited by any particular theory or mechanism, it is envisioned that a non-peptide bond located at one or both of the peptide termini reduces or eliminates degradation of the peptide analog by circulating processive exopeptidases, and prolongs the half-life of the copolymer following administration to a subject.

The peptide analogs like the peptide parents are synthesized by chemical methods which are well known in the art and are described further in Merrifield, J. (1969) J. Am. Chem. Soc. 91: 501; Chaiken I. M. (1981) CRC Crit. Rev. Biochem. 11:255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing, which are incorporated herein by reference). Suitable fmoc and tboc precursors for introduction of non-peptide bonds, such as olefin bonds, are available from PharmaCore, Inc. (High Point, N.C.), and from AnaSpec, Inc. (San Jose, Calif.), and can also be obtained by custom order synthesis.

Systematic introduction into the peptide of non-peptide bonds at either of the C- or the N-termini, or at a position adjacent to a terminus, can be achieved by using, for example, an fmoc- or tboc-protected immobilized (solid state) amino acid analog precursor, or by using an initially supplied liquid state amino acid analog precursor, to form an initial non-peptide bond. Alternatively, the amino acid analog precursor can be supplied at the end of the reaction, to participate in formation of terminal non-peptide bonds. Formation of interior non-peptide bonds, located distal to the termini, can be achieved by adding a limiting amount of peptide analog precursor at an intermediate time point in the synthesis process.

Therapeutic Compositions in the Methods of the Invention

A pharmaceutically acceptable carrier includes any and all solvents, dispersion media, coatings, antimicrobials such as antibacterial and antifungal agents, isotonic and absorption delaying agents and the like that are physiologically compatible. Preferably, the carrier is suitable for intravenous, intramuscular, oral, intraperitoneal, transdermal, or subcutaneous administration, and the active compound can be coated in a material to protect it from inactivation by the action of acids or other adverse natural conditions.

The methods of the invention include incorporation of a peptide as provided herein into a pharmaceutical composition suitable for administration to a subject. A composition of the present invention can be administered by a variety of methods known in the art as will be appreciated by the skilled artisan. The active compound can be prepared with carriers that will protect it against rapid release, such as a controlled release formulation, including implants, transdermal patches, and microencapsulated delivery systems. Many methods for the preparation of such formulations are patented and are generally known to those skilled in the art. See, e.g., Sustained and Controlled Release Drug Delivery Systems, J. R. Robinson, Ed., Marcel Dekker, Inc., NY, 1978. Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration.

Dosage regimens can be adjusted to provide the optimum desired response (e.g., a therapeutic response). For example, a single bolus or oral dose can be administered, several divided doses can be administered over time, or the dose can be proportionally reduced or increased as indicated by the exigencies of the disease situation.

In general, an embodiment of the invention is to administer a suitable daily dose of a therapeutic peptide composition that will be the lowest effective dose to produce a therapeutic effect, for example, mitigation of symptoms. The therapeutic peptide compounds of the invention are preferably administered at a dose per subject per day of at least about 2 mg, at least about 5 mg, at least about 10 mg or at least about 20 mg as appropriate minimal starting dosages. In general, the compound of the effective dose of the composition of the invention can be administered in the range of about 50 to about 400 micrograms of the compound per kilogram of the subject per day.

A physician or veterinarian having ordinary skill in the art can readily determine and prescribe the effective dose of the pharmaceutical composition required. For example, the physician or veterinarian could start doses of the compound of the invention employed in the pharmaceutical composition at a level lower than that required in order to achieve the desired therapeutic effect, and increase the dosage with time until the desired effect is achieved.

In another embodiment, the pharmaceutical composition includes also an additional therapeutic agent. Thus in a method of the invention the pharmaceutical peptide composition can be administered as part of a combination therapy, i.e. in combination with an additional agent or agents. Examples of materials that can be used as combination therapeutics with the peptide for treatment of autoimmune disease as additional therapeutic agents include: an antibody or an antibody fragment that can bind specifically to an inflammatory molecule or an unwanted cytokine such as interleukin-6, interleukin-8, granulocyte macrophage colony stimulating factor, and tumor necrosis factor-α; an enzyme inhibitor which can be a protein, such as α₁-antitrypsin, or aprotinin; an enzyme inhibitor which can be a cyclooxygenase inhibitor; an engineered binding protein, for example, an engineered protein that is a protease inhibitor such an engineered inhibitor of a kallikrein; an antibacterial agent, which can be an antibiotic such as amoxicillin, rifampicin, erythromycin; an antiviral agent, which can be a low molecular weight chemical, such as acyclovir; a steroid, for example a corticosteroid, or a sex steroid such as progesterone; a non-steroidal anti-inflammatory agent such as aspirin, ibuprofen, or acetaminophen; an anti-cancer agent such as methotrexate, cis-platin, 5-fluorouracil, or adriamycin; a cytokine blocking agent; an adhesion molecule blocking agent; or a cytokine.

An additional therapeutic agent can be a cytokine, which as used herein includes without limitation agents which are naturally occurring proteins or variants and which function as growth factors, lymphokines, interferons particularly interferon-β, tumor necrosis factors, angiogenic or antiangiogenic factors, erythropoietins, thrombopoietins, interleukins, maturation factors, chemotactic proteins, or the like. An additional agent to be added to a copolymer of amino acids which are embodiments of the invention herein can be a different copolymer, for example, Copaxone® which is a YEAK or Cop 1, or a copolymer comprising a subset of these or other amino acids (Aharoni et al. WO 00/05250, PCT/US99/16747), or an oligopeptide or peptide derivative (Strominger et al. WO 00/05249, PCT/US99/16617; WO 02/59143, PCT/US02/02071). Preferred therapeutic agents to be used in combination with a composition of the invention and which are cytokines include interferon-β, interleukin-4 and interleukin-10.

A therapeutic agent to be used with the composition of the invention can be an engineered binding protein, known to one of skill in the art of remodeling a protein that is covalently attached to a virion coat protein by virtue of genetic fusion (Ladner, R. et al., U.S. Pat. No. 5,233,409; Ladner, R. et al., U.S. Pat. No. 5,403,484), and can be made according to methods known in the art. A protein that binds any of a variety of other targets can be engineered and used in the present invention as a therapeutic agent in combination with a heteropolymer of the invention.

An improvement in the symptoms as a result of such administration is noted by a decrease in frequency of recurrences of episodes of the autoimmune condition such as MS, by decrease in severity of symptoms, and by elimination of recurrent episodes for a period of time after the start of administration. A therapeutically effective dosage preferably reduces symptoms and frequency of recurrences by at least about 20%, for example, by at least about 40%, by at least about 60%, and by at least about 80%, or by about 100% elimination of one or more symptoms, or elimination of recurrences of the autoimmune disease, relative to untreated subjects. The period of time can be at least about one month, at least about six months, or at least about one year.

Methods of use of peptides having sequences provided herein can be the basis of treating other autoimmune diseases which are associated with HLA-DR gene products, by competing with candidate autoantigens for binding to these protein receptor molecules, or by inducing T cell anergy or even T cell apoptosis, or by suppression of T cells, such that subsequent T cell response to an autoantigen is inhibited in vivo. Further, peptides having within the sequence one or more additional components, such as amino acid analogs or derivatives added in varying quantities into the polymerization reaction, can be effective inhibitors of a variety of autoimmune T cell responses.

The activity of Cop1 appears to involve, as a first step, binding to the surface of antigen-presenting cells (APC), for example to class II MHC proteins (Fridkis-Hareli M. et al. 1994. Proc. Natl. Acad. Sci. USA 91:4872-4876), following which its effectiveness may be due either to competition with myelin antigens (for example, MBP, PLP, MOG) for activation of specific effector T cells recognizing peptide epitopes derived from these proteins (Ben-Nun, A. et al. 1996. J. Neurol. 243:S14-22; Teitelbaum, D. et al. 1996. J. Neuroimmunol. 64:209-217), and/or induction of antigen-specific regulatory T cells (Aharoni R. et al. 1993. Eur. J. Immunol. 23:17-25).

Examination of additional therapeutic agents and investigation of the mechanisms involved in their activities could potentially result in information that could lead to improved therapeutic reagents. Recent studies have shown that virtually all of the large variety of copolymers found in the random mixture of YEAK bound to purified molecules of each of human HLA-DR1, -DR-2 and -DR4 molecules, showing that YEAK generally binds to purified class II MHC proteins (Fridkis-Hareli, M., and J. L. Strominger, 1998. J. Immunol. 160:4386-4397). Cop1 further competes for binding of MBP 85-99 to HLA-DR-2 (DRB1*1501) and inhibits responses of DR-2-restricted T cells to MBP 85-99. Study of the binding to class II MHC molecules of random copolymers containing only 3 of the 4 amino acids of Cop1, for example, YAK, revealed that YAK is the most effective (Fridkis-Hareli, M. et al. 1999. Int. Immunol. 11:635-641).

The binding motif of Cop1 to the MS-associated molecule HLA DR-2 (DRB1*1501) shows E at P-2, K at P−1 and Y at P1, with no preferences observed at other positions (Fridkis-Hareli, M. et al. 1999. J. Immunol. 162:4697-4704). Further, A is overrepresented at P1. As P1 is the anchor position, binding of Y at this position was not anticipated. The P1 pocket in proteins encoded by the DR-2 allele is small (due to the presence of β86Val rather than β86Gly), and overrepresentation of A at this position may result from this fact. The effect of K at P−1 appears to be due to stabilization of binding by the interaction of K with residues in the top of the α1 helix, similarly to residue K at P−1 of HA 306-318 (SEQ ID NO: 5) complexed with HLA-DR1 which can interact with the side chains of α1 helix residues at Sα53 or Eα55 (Stem, L. J. et al. 1994. Nature 368:215-221).

Those skilled in the art will recognize or be able to ascertain using routine experimentation the numerous equivalents to the examples and claims herein, which are exemplary and are not intended to be further limiting. The contents of all references cited throughout the application are hereby incorporated by reference. A portion of the invention appeared in Stern, J. N. H. et al., Proc Natl Acad Sci U.S.A. 102(5):1620, published Feb. 1, 2005, and online Jan. 21, 2005, and which is hereby incorporated in its entirety by reference herein.

### EXAMPLES

The following Materials and Methods were used throughout the examples.

Mice

Humanized mice previously described (Stem, J. N. et al. 2004. PNAS 101:11743) are similar to another double-transgenic mouse (Madsen, L. S. et al. 1999. Nat Genet 23:343), was developed independently. SJL/J (H-2^{s}) mice were purchased from the Jackson Laboratory (Bar Harbor, Me.). Eight to 12 week old, female mice were used in all experiments. Animals were maintained at the animal facilities of Harvard University according to the animal protocol guidelines of Harvard Medical School, Boston, Mass.

Peptide Synthesis

Peptides were synthesized as described previously (Fridkis-Hareli, M. et al. 2001 Hum Immunol 62:753). A synthetic peptide having a sequence from a neuraminidase (Nase) peptide having residues 101-120, EALVRQGLAKVAYVYKPNNT (SEQ ID NO: 27) was used as a negative control in T cell proliferation experiments.

Protein Expression and Purification

Examples used methods previously described (Fridkis-Hareli, M. et al. 2001 Hum Immunol 62:753) with LB3.1 to isolate soluble HLA-DR2 molecules which had been expressed in Drosophila S2 cells and were purified by affinity chromatography (Smith, K. J. et al. 1998. J Exp Med 188:1511). Binding of biotinylated MBP85-99 to HLA-DR2 in competition with peptides or MBP 85-99 was analyzed.

Competitive Binding Assay for HLA-DR2 (DRA/DRB1*1501) Protein

These studies were performed as described (Fridkis-Hareli, M. et al. 2002. J Clin Invest 109:1635). Percent inhibition was determined by using the following formula: (Absorbance in control samples containing biotinylated MBP 85-99)-(Absorbance in samples containing unlabelled MBP 85-99 or the novel peptides)/(Absorbance in samples containing unlabelled MBP 85-99 or the novel peptides)×100. Antigen presentation assay was performed as described previously [Fridkis-Hareli, M. et al. 2001 Hum Immunol 62:753].

Sorting by Flow Cytometry of Transgenic (tg) and Endogenous (non-tg) T Cells from Humanized Mice

To sort human TCR transgenic T cells specific to MBP 85-99, splenocytes from humanized mice were incubated with anti-CD4-APC (clone GK1.5, obtained from PharMingen, San Diego, Calif.) and anti-human Vβ2-PE (Immunotech, Marseille, France) at room temperature for 20 min. After washing twice, tg and endogenous or non-tg CD4 T cells (hVβ2⁻CD4 cells) were sorted using a FACS Vantage SE apparatus (Becton Dickinson, San Diego, Calif.). The purity of fractionated hVβ2⁺CD4⁺ and hVβ2⁻CD4⁺ T cells was determined to be 98-99%.

Generation of Peptide-Specific Short-Term T Cell Lines and Analysis of Proliferative Responses to Peptides

SJL/J mice were immunized with PLP 139-151 or with each of the peptides (100 μg per mouse) emulsified in complete Freund's adjuvant (CFA; Difco Laboratories, Detroit, Mich.). Ten days later, single cell suspensions were prepared from spleens and lymph nodes, and were stimulated and re-stimulated with corresponding peptides at a concentration of 10 μg/ml in the presence of APC. After two rounds of stimulation, viable lymphocytes were separated by Ficoll-Hypaque density gradient centrifugation and were tested for specificity at a dose range of 0 to 50 μg/ml. NASE 101-120 peptide, which also binds I-A^{s} was used as a negative control. Lymphocytes were cultured in 96-well plates at a density of 5×10⁵ cells per ml for 48 hrs, and were pulsed with ³[H]-thymidine, 1 μCi per well. After sixteen hours of culture, plates were harvested and a Wallac scintillation counter (model 1250) was used to analyze the proliferative response determined by amount of ³[H]-thymidine incorporation CPM. To determine the proliferative response of each of tg T cells expressing HLA-DR2 and human MBP 85-99-specific TCR, and non-tg T cells, the cells fractionated by flow cytometry were seeded in complete medium at 4×10⁵ cells/well in 96-well U-bottom microtiter plates. Irradiated splenocytes of humanized mice were used as APC. Cultures were stimulated with 10 μg/ml of MBP 85-99 or with 100 μg/ml of each of peptides J2, J3, and J5, and the proliferative responses were measured as described above.

Cytokine Measurement by ELISA

Lymphocytes from SJL/J mice immunized with PLP 139-151 or with each of peptides J2, J3, and J5 were re-stimulated with corresponding peptides at a concentration of 10 μg per ml in the presence of APC in 24-well plates for 2 days. Cytokine mAb directed against each of IL-2, IL-4, IL-10, and IFN-γ was coated into wells of 96-well plates at a concentration of 1 μg/ml overnight. Plates were washed and were treated with blocking solution (Kierkegaard and Perry, Gaithersburg, Md.), followed by incubation of each of the cytokine standards and the culture supernatants overnight at 4° C. After washing, plates were incubated with corresponding biotinylated anti-cytokine-detecting mAb (1 μg/ml) for 2 hrs. Plates were developed by addition of avidin/peroxidase and its substrate. The mAb pairs used were from the following clones: IL-2: JES6-A112, and JES6-5H4; IL-4: 11B11 and BVD6-24G2; IL-10: JES5-16E3 and SXC-1; and IFN-γ: R4-6A2 and XMG 1.2 (PharMingen).

Effect of Novel Peptides on Induction of EAE

To determine the therapeutic effect of peptides on appearance and progression of the mouse model diseases EAE, a humanized mouse model was adopted. Eight to 12 week old female humanized mice were immunized subcutaneously (s.c.), each with 150 μg of MBP 85-99, or were co-immunized with 150 μg MBP 85-99 and 150 μg of each of the peptides, emulsified in CFA. Pertussis toxin, 150 ng (List Biological Laboratories, Campbell, Calif.), was administered intraperitoneally on the day of immunization and on day 2 post-immunization. The mice were monitored for appearance of clinical signs of EAE, and were scored from 0-5 as follows: 1, limp tail; 2, hind limb paresis; 3, complete hind limp paralysis; 4, four limbs paralyzed; 5, moribund.

Adoptive Transfer of Peptide-Specific T Cell Lines

SJL/J mice were immunized with either 50 μg PLP 139-151 peptide or with 250 μg of each of peptides J2, J3, or J5. Stimulator cells were prepared by loading respective samples of naïve SJL/J splenocytes in vitro for 12 hours with 10 μg/ml of each of J2, J3 or J5. Ten days post immunization, splenocytes were co-cultured with irradiated stimulators (3000 rad) in 1:1 ratio for five days in T 25 flasks (1×10⁸ total cells in medium containing 20 units of IL-2) and were re-stimulated weekly for 3 weeks with fresh antigen loaded splenocytes to obtain cell lines. T cells from these lines 5×10⁶ were injected intravenously into naïve 6-8 week old SJL/J mice, and the protocol described above was followed.

Detection of PLP 139-151-Reactive CD4⁺ T Cells by Using I-A^{s}/PLP 139-151 Tetramers

SJL/J mice in groups of 3 animals were immunized with CFA alone or with PLP 139-151, with or without each of the peptides (75 μg each/mouse) in CFA. On day 10, all mice were sacrificed, and single cell suspensions were prepared and cultured at a density of 5×10⁶ cells per ml in DMEM medium containing 10% fetal calf serum (FCS), 2 mM L-glutamine, 10 mM HEPES, 2 mM mercaptoethanol, penicillin (1000 U/ml) and streptomycin (1 μg/ml) (Biowhittaker, Walkersville, Md.). The cultures from each of the above groups were re-stimulated with PLP 139-151 with or without peptides (25 μg per ml). The mice treated with CFA only were used as negative control to validate immunizations; the PLP 139-151-immunized mice were used as positive controls to which the effect of peptides on PLP 139-151-specific CD4⁺ T cell responses was compared.

After 4 days of culturing, cell suspensions were subjected to Ficoll-Hypaque density gradient centrifugation and were washed twice using PBS. Approximately 1×10⁷ cells per ml were incubated with I-A^{s} tetramers (PLP 139-151 or Theiler's murine encephalomyelitis virus (TMEV) 70-86) conjugated with phycoerythrin (PE) in DMEM medium supplemented with interleukin (L)-2 at 37° C. for 3 hrs at a concentration of 30 μg per ml, as described in methods (Reddy, J. et al. 2003. J Immunol 170:870). Cells were washed with 4 ml of PBS buffer containing 2% FCS and 0.1% sodium azide, and were stained with anti-CD25-FITC (clone 7D4), anti-CD4-APC (clone RM4.5) and 7-AAD (PharMingen, San Diego, Calif.). Cells were incubated at room temperature for 20 min., were washed as above, and were sorted using a FACSort flow cytometer (Becton and Dickinson, San Jose, Calif.).

### Example 1

### Design of Synthetic Peptide 15mers

The design of peptide 15mers was based on the following criteria: MBP85-99 has V89 at P1 and F92 at P4. Further, a Y at the P1 of peptides or copolymers (as found in Copolymer 1) is too large for the P1 pocket. Such peptides are therefore poor binders to HLA-DR2. Further, an F residue at P1 is less effective than V (Wucherpfennig, K. W. et al. 1994. J Exp Med 179:279; Fridkis-Hareli, M. et al. 2002. J Clin Invest 109:1635). A peptide having a large hydrophobic amino acid W replaced by F at P4 of MBP85-99 was found to effectively stimulate an HLA-DR2 restricted T cell clone (Wucherpfennig, K. W. et al. 1994. J Exp Med 179:279; Smith, K. J. et al. 1998. J Exp Med 188:1511). Moreover, in the binding motif of Cop1 to HLA-DR2, Y was eluted from the P4 pocket (Fridkis-Hareli, M. et al. 1999. J Immunol 162:4697). Therefore for the design of peptides herein, both the P1 and P4 were designed to have hydrophobic amino acid residues of different sizes V, F, or Y (Table 1). In addition, K at P5, the major TCR contact residue in MBP85-99, was used in all peptides. Furthermore, the residues corresponding to P6 to P15 in MBP85-99 were occupied by alanine (A), except that in some peptides, prolines (P) were placed at the penultimate position P10, since proline residues are resistant to exopeptidases in serum. For the purpose of in vivo EAE studies a P residue at P−5 and at P−6 were also added to some peptides for the same reason. These prolines did not change the binding affinity of peptides for HLA-DR2 (Table 1) compared to affinity of the peptides lacking prolines at such positions. The TCR contact site V88 of MBP85-99 at P−1 (Smith, K. J. et al. 1998. J Exp Med 188:1511) was replaced by lysine; this modification was hypothesized to enhance binding to HLA-DR2 (Fridkis-Hareli, M. et al. 2001 Hum Immunol 62:753). These peptides were designed to achieve a minimum possible homology with natural antigen MBP85-99, yet bind with high affinity to HLA-DR2.

### Example 2

### Inhibition of Binding of MBP85-99 to HLA-DR2 (DRA/DRB1*1501) by Synthetic Peptides

Extent of competition between each of the peptides and the immunodominant epitope MBP85-99 for binding to HLA-DR2 molecules was determined. Several peptides were found to inhibit binding of biotinylated MBP 85-99 to HLA-DR2 molecules very efficiently (FIG. 1A). The most effective peptides were found to be J3, J5, J8, J9, J13, J15 and J17. All of these had V or F at position P1, and Y or F at position P4 (see amino acid sequences in Table 1). Cop1 inhibited binding of biotinylated MBP85-99 less efficiently than peptides J1, J2, J3 and J5. (Table 1 and FIG. 1A). All the other peptides had comparable inhibition at the lowest concentrations used (1.5 μM). Surprisingly, peptides designed to have E at P1, as a control, were found to have significantly decreased binding (J4 and J6, Table 1 and FIG. 1B). Notably, Cop1 which contains E as well as Y, had a lower binding affinity for HLA-DR2 (FIG. 1A). Peptides J3 and J5 showed maximum inhibition of MBP85-99 binding to HLA-DR2 molecules, particularly at lower concentrations (FIG. 1A). Peptides J12 and J13 of the set J11-J17, differing only in the order of the amino acids at the second and third positions, showed similar binding of DR2 (Table 1).

### Example 3

### Inhibition by Synthetic Peptide 15mers of IL-2 Secretion by an HLA-DR2-Restricted, MBP85-99-Specific T-Cell Hybridoma

The findings herein that the peptides competed with MBP85-99 in binding to HLA-DR2 provided a basis to test the hypothesis that the peptides also might inhibit MBP-specific T cells. The T cell hybridoma 8073 that had been transfected with MBP85-99-specific TCR, from clone (ObA.12) derived from a patient with relapsing-remitting MS carrying HLA-DR2 (Madsen, L. S. et al. 1999. Nat Genet. 23:343) was used for this assay. Peptides J1, J2, J3, J4, and J5 were used to inhibit IL-2 secretion, as measured by proliferation of the IL-2-responsive CTLL line, and were compared to Cop1 (FIG. 1B). Peptide J1, J2, J3 and J5 inhibited proliferation of MBP 85-99-specific T cell hybridoma 8073 in a dose-dependant manner. (FIG. 1B). This inhibition was significantly greater than the effect of either Cop1 or J4 (FIG. 1B; p); ≦0.05).

Peptide J5 was the best inhibitor, as well as the best binder, indicating that the V at P1 and the Y at P4 were significant for enhanced binding of these peptides to HLA-DR2. Inhibition by the peptides of IL-2 production by the 2E12 T cell line obtained from a double transgenic mouse (Madsen, L. S. et al. 1999. Nat Genet. 23:343) was comparable to those obtained with the 8073 T cell hybridoma.

### Example 4

### Synthetic Peptide 15mers are Immunogenic

To examine immunogenicity of the synthetic peptides, humanized mice were immunized with 100 μg each of J2, J3, J5 or MBP85-99. Spleen and lymph node cells were collected 10 days later and were stimulated in vitro with the corresponding peptides. It was found herein that MBP85-99 elicited a strong proliferative response (FIG. 2A, top panel), i.e., this peptide was strongly immunogenic. Likewise, in the mice immunized with either J3 or J5 alone, a lower response was induced, showing by analogy to responses to copolymers (Illés, Z. et al. 2004. PNAS 101:11749) that the non-transgenic (tg) endogenous T cells responded to these peptides as presented by HLA-DR2. However, a negligible response to J2 (that bound weakly) was seen (FIG. 2A, second panel).

Immunogenicity of these peptides was further verified in SJL/J mice (in which I-A^{s} is the only expressed class II MHC protein; FIG. 2B) and compared to PLP 139-151 (positive control), one of the most immunodominant and encephalitogenic epitopes of PLP in SJL/J mice (Tuohy, V. K. et al. 1989. J Immunol 142:1523, Kuchroo, V. K., et al. 1991. Pathobiology 59:305). Cells from SJL/J mice immunized with these peptides were able to mount an immune response following re-stimulation by the synthetic peptide 15mer (J2, J3 or J5; FIG. 2B, second, third and fourth panels).

Thus, the peptides were found to be able to bind both to HLA-DR2 and to I-A^{s}, and to induce peptide-specific T cells.

### Example 5

### Peptide 15mer-Reactive T Cell Lines do not Cross-React with Those of Natural Ligands

As shown with copolymers (Stern, J. N. et al. 2004. PNAS 101:11743), transgenic T cells respond to MBP85-99 with a smaller response than that of endogenous T cells. Therefore, MBP85-99-specific transgenic (hVβ2⁺CD4⁺) and endogenous (non-transgenic, hVβ2⁻CD4⁺) T cells were sorted by flow cytometry from spleen cells of humanized double transgenic mice. Both sets were stimulated with 10 μg/ml MBP85-99 or with 100 μg/ml of each of J2, J3, or J5 in the presence of irradiated syngeneic splenocytes.

It was found that MBP85-99 efficiently stimulated the transgenic CD4⁺ cells to an extent that was significantly higher than that obtained from endogenous CD4⁺ cells (FIG. 3A, bars on left). By contrast, J2, J3, and J5 did not induce proliferation of either MBP85-99-specific transgenic or endogenous CD4⁺ cells, confirming that these peptides do not cross-react with this human MBP85-99-specific TCR or with the non-clonal endogenous TCR that respond to MBP85-99 (FIG. 3A, bars on right; no proliferation observed). The lack of response to J2, J3 or J5 was not due to cell death, since in all of the treatment groups, the numbers of viable cells observed were comparable. Altered peptide ligands (APLs) of MBP 85-99 (four amino acid replacements) were previously shown to cross-react with the natural ligand. This cross reaction was proposed to be a mechanism by which those APLs exacerbated the disease in MS patients (Kappos, L. et al. 2000. Nat Med 6:1176, Bielekova, B. et al. 2000. Nat Med 6:1167).

Lack of cross reactivity to the peptides herein was further verified by using a PLP139-151 specific cell line. This line responded only to PLP 139-151 and not to the control peptide, Staphylococcus aureus nuclease (Nase)101-120, nor to any of J2, J3 or J5 15mers (FIG. 3B). Similarly, T cell lines derived from SJL/J mice immunized with each of J2, J3 or J5, responded only to the cognate peptide, and not to PLP139-151.

### Example 6

### Peptide 15mer-Specific T Cells Produce Th2 Cytokines Preferentially

The cytokine profiles of short term lines that had been stimulated with each of J2, J3 or J5 were compared with those of PLP139-151-stimulated cultures (FIG. 4). SJL/J mice were immunized with PLP139-151, J2, J3 or J5 in CFA, and spleen and lymph node cells were stimulated ten days later with the cognate peptides in the presence of irradiated antigen presenting cells (see Methods) for 2 days. In cultures stimulated with PLP139-151, it was observed that L-2 and IFN-γ were produced in significant amounts (bars on the left of each panel), while neither was produced in those stimulated with the peptide 15mers (FIG. 4). In contrast, IL-10 production was produced in cultures stimulated with peptides J3 and J5 in amounts about three fold higher than in those stimulated with PLP139-151 or peptide J2. Secretion of IL-4 was comparable in all of the cultures regardless of peptide used.

### Example 7

### J2, J3 and J5 Peptides Ameliorate EAE Induced by MBP 85-99 in Humanized Mice or in PLP 139-151 SJL/J Mice

The ability of each of J2, J3 and J5 to ameliorate the severity of mouse model disease EAE in vivo was tested in two models of EAE: MBP85-99 induced EAE in HLA-DR2 and MBP85-99-specific TCR double tg mice; or PLP139-151 induced EAE in SJL/J mice.

Results in the control “untreated” humanized mice after immunization with MBP85-99, show that to induce EAE, all mice in the group developed clinical signs of EAE between day 6 and day 9 (FIG. 5A). One mouse died, and the remaining mice entered a chronic phase of the disease with a mean symptom score of 2.0 that reached a mean maximum score of 2.5.

In contrast, in mice co-immunized with each of the peptides J2, J3 or J5 and with MBP 85-99, the onset of disease was delayed by 2-5 days and the disease severity was strikingly reduced during the acute phase of EAE (FIG. 5A). The maximum mean scores of the mice treated with J2, J3, or with J5 were reduced to about 1 (limp tail) compared to the score for untreated mice. Importantly, no mortalities occurred in any of the co-immunized groups. In a previous study, Cop1 reduced the signs of EAE but less effectively, to a mean maximal score of 3.5 (Illés, Z. et al. 2004. PNAS 101:11749).

EAE was induced in SJL/J mice by immunizing with PLP139-151. The first sign of EAE appeared at day 7 and reached a maximum mean score of 3 by day 16 (FIG. 5B). Three of the eight mice immunized with PLP139-151 died. The severity of EAE in groups of six mice co-immunized with Cop1 or with each of J2, J3, and J5 together with PLP139-151 was moderately suppressed by Cop1 (mean maximum score 2.5 at around day 22, followed by slow recovery; FIG. 5B, open diamonds). In contrast, mice co-immunized with any of the peptides J2, J3 or J5 developed only a mild disease (mean clinical score approximately 1; FIG. 5B open squares, open triangles, and open circles, respectively). No mortalities were observed for mice in any of these peptide-treated groups. Thus, the severity of EAE in J2, J3 or J5 co-immunized groups was significantly lower than in mice immunized with PLP139-151 or co-immunized with Cop1.

### Example 8

### Suppression by Adoptive Transfer of Peptide-Specific T Cell Lines

SJL/J mice were immunized subcutaneously with either 50 μg of PLP 139-151 or with 250 μg of one of peptides J2, J3, or J5. Starting on day 10, splenocytes of these mice were stimulated three times in vitro every other week with peptide-pulsed irradiated splenocytes from naïve mice. After the third round of stimulation, 92% CD3⁺ T cells were obtained. Then, a sample of 5×10⁶ T cells of the J2, J3, or J5 stimulated population was administered intravenously to each mouse. EAE was induced the next day by administering PLP 139-151 in CFA.

The data show that the mice that received peptide-specific T cells (FIG. 6, open symbols) had ameliorated symptoms: both a delayed onset and a much milder form of EAE, compared to PLP 139-151 controls (FIG. 6, closed symbols), was observed in mice that were administered peptide-specific T cells.

### Example 9

### Peptide 15mers Inhibit the Expansion of PLP 139-151-Specific T Cells

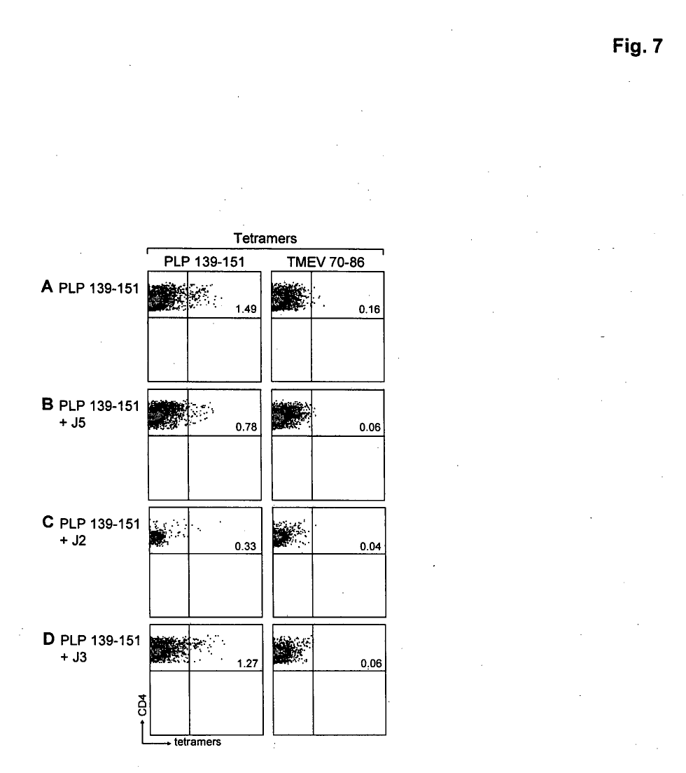

To determine whether the peptides J2, J3, and J5 affect the expansion of antigen-specific T cells, I-A^{s}/PLP139-151 tetramers were used. Lymphocytes from SJL mice immunized either with PLP139-151 or co-immunized with each of these peptides and with PLP139-151 were obtained, were cultured with the PLP139-151 for 4 days, and samples were analyzed by cell sorting for number of PLP tetramer reactive cells. Data are shown in FIG. 7, left panels. A peptide from TMEV capsid protein was used as a negative control (FIG. 7, right).

It was found that in SJL mice immunized with PLP139-151, expansion of PLP tetramer reactive cells was observed (1.5%). Further, this response was specific, since there was no response to TMEV control tetramers (FIG. 7A, comparing right and left top panels).

In striking contrast, expansion of PLP139-151-specific T cells was reduced markedly in mice co-immunized with PLP139-151 and either novel peptide J5 or J2 (FIG. 7, panels B and C).

TABLE 1

| | |
|---|---|
| peptide/copolymer. | % inhibition* |

| | | |
|---|---|---|
| Cop1 | poly[Y, E, A, K]n | 8 |
| | −4 1 5 10 | |
| | \| \| \| \| | |
| J1 | EKAKYEAYKAAAAAA | 18 |
| J2 | EKPKYEAYKAAAAPA | 14 |
| J3 | EKPKFEAYKAAAAPA | 26 |
| J4 | EKAKEEAYKAAAAAA | 0 |
| J5 | EKPKVEAYKAAAAPA | 41 |
| J6 | EKPKEEAFKAAAAPA | 0 |
| J7 | EKAKFEAFKAAAAAA | 19 |
| J8 | APEKAKFEAFKAAAAPA | 34 |
| J9 | APEKAKFEAYKAAAAPA | 31 |
| J10 | APEKAKVEAFKAAAAPA | 5 |
| J11 | EAKKYEAYKAAAAAA | 0 |
| J12 | EAPKFEAYKAAAAPA | 13 |
| J13 | EAPKVEAYKAAAAPA | 44 |
| J14 | EAPKFEAFKAAAAPA | 15 |
| J15 | APEAKKFEAFKAAAAPA | 26 |
| J16 | APEAKKFEAYKAAAAPA | 14 |
| J17 | APEAKKVEAFKAAAAPA | 23 |
| MBP85-99 | ENPVVHFFKNIVTPR | 15 |

*Binding of biotinylated MBP (0.5 μM) by peptide competitor (1 μM)

1-96. (canceled)

97. A composition having a peptide comprising an amino acid sequence selected from the group consisting of: EEAYK (SEQ ID NO: 21), VEAFK (SEQ ID NO: 25), and YEAFK (SEQ ID NO: 26), wherein the peptide is at least 6 amino acid residues in length; EKAKEEAYKAAAAAA (SEQ ID NO:4), APEKAKVEAFKAAAAPA (SEQ ID NO: 10), APEAKKFEAFKAAAAPA (SEQ ID NO: 15), APEAKKFEAYKAAAAPA (SEQ ID NO: 16), and APEAKKVEAFKAAAAPA (SEQ ID NO: 17); and, EKAKEEAYK (SEQ ID NO: 30), EKAKFEAYK (SEQ ID NO: 34), EKAKVEAFK (SEQ ID NO: 35), EAPKFEAYK (SEQ ID NO: 37), EAPKFEAFK (SEQ ID NO: 39), EAKKFEAFK (SEQ ID NO: 40), and EAKKFEAYK (SEQ ID NO: 41).

98. A composition according to claim 97, wherein the amino acid sequence of the peptide further contains at least one alanine residue (A) at a terminus of the peptide, wherein the terminus is selected from the group consisting of an amino terminus and a carboxy terminus.

99. The composition according to claim 98, wherein the at least one (A) residue is a plurality of (A) residues further contains at least one proline (P) residue at a position that is penultimate to the amino terminus or the carboxy terminus of the peptide.

100. A composition having a peptide consisting essentially of an amino acid sequence selected from the group consisting of: EKAKEEAYKAAAAAA (SEQ ID NO: 4), APEKAKVEAFKAAAAPA (SEQ ID NO: 10), APEAKKFEAFKAAAAPA (SEQ ID NO: 15), APEAKKFEAYKAAAAPA (SEQ ID NO: 16), and APEAKKVEAFKAAAAPA (SEQ ID NO: 17).

101. The composition comprising a plurality of peptides according to claim 97.

102. The composition according to claim 97, wherein a terminal amino acid residue is substituted, wherein the terminal is an amino terminal or a carboxy terminal.

103. The composition according to claim 102, wherein the substituted terminal amino acid acetylated, methylated, formylated, or t-butyloxylated.

104. The composition according to claim 102, wherein the carboxy terminal amino acid residue is methylated or amidated.

105. The composition according to claim 97, wherein an internal amino acid residue is substituted.

106. The composition according to claim 105, wherein the internal amino acid residue is a lysine (K), and is substituted at the ε-amino group.

107. The composition according to claim 97, wherein at least one peptide bond is substituted by a non-peptide bond.

108. The composition according to claim 107, wherein the non-peptide bond is selected from the group consisting of: —CH₂NH—, —CH₂S—, —CH₂CH₂—, —CH═CH—, —COCH₂—, —CH(OH)CH₂—, and —CH₂SO—.

109. The composition according to claim 97, wherein at least one amino acid residue is an amino acid residue analog.

110. The composition according to claim 109, wherein the amino acid residue analog is selected from the group consisting of: a D-amino acid, an alkylated amino acid, a halogenated amino acid, and an amino acid substituted with a non-naturally occurring side chain.

111. A pharmaceutical composition comprising at least one peptide from the group of peptides having amino acid sequences: EKAKEEAYKAAAAAA (SEQ ID NO: 4), APEKAKVEAFKAAAAPA (SEQ ID NO: 10), APEAKKFEAFKAAAAPA (SEQ ID NO: 15), APEAKKFEAYKAAAAPA (SEQ ID NO: 16), and APEAKKVEAFKAAAAPA (SEQ ID NO: 17) in an effective dose.

112. The compositions according to claim 111, wherein the peptide length is at least one length selected from the group of: at least 6 amino acid residues, at least 15 amino acid residues, and at least 75 amino acid residues.

113. The composition according to claim 112, wherein the peptides have a length of at least 75 amino acid residues and the amino acid sequence in the peptide in the composition is present in at least two iterations.

114. The composition according to claim 111, further comprising a pharmaceutically acceptable salt or buffer.

115. The composition according to either of claims 97 or 111 wherein the peptide is capable of binding to class II MHC protein HLA-DR2.

116. The composition according to either of claims 97 or 111 present in a unit dose effective for treatment of a subject for an autoimmune condition.

117. The composition according to either of claims 97 or 111, wherein the autoimmune condition is a cell mediated disease.

118. The composition according to either of claims 97 or 111, wherein the autoimmune condition is an antibody mediated disease.

119. The composition according to claim 117, wherein the autoimmune condition is a demyelinating condition.

120. The composition according to claim 117, wherein the autoimmune condition is mediated by a T cell or a natural killer (NK) cell.

121. The composition according to claim 117, wherein the autoimmune condition is selected from the group consisting of diabetes, multiple sclerosis, and Hashimoto's thyroiditis.

122. The composition according to claim 119, wherein the demyelinating condition is multiple sclerosis (MS).

123. The composition according to claim 118, wherein the condition is systemic lupus erythematosus (SLE) or myasthenia gravis.

124. The composition according to either of claims 97 or 111, further comprising a pharmaceutically acceptable carrier.

125. The composition according to either of claims 97 or 111, wherein the peptide is orally available.

126. An isolated peptide composition having an amino acid sequence capable of inhibiting an immune response in a subject to an autoantigen associated with multiple sclerosis, wherein a position in the amino acid sequence of the peptide that corresponds to an antigen binding pocket in a peptide binding groove of an class II MHC protein HLA-DR2 is identified as a particular amino acid, wherein the amino acid residue in the position of the sequence that corresponds to the P1 pocket in peptide binding groove of the MHC protein is selected from the group consisting of a tyrosine, a valine, a tryptophan and a phenylalanine; and wherein the amino acid residue in the position that corresponds to the P4 pocket is a large hydrophobic amino acid selected from the group consisting of a methionine, a phenylalanine, a tyrosine, and a tryptophan; and wherein the amino acid residue located eight residues beyond (P9 pocket) the first amino acid position of the sequence that corresponds to the P1 pocket in the MHC class II peptide binding groove is a small neutral amino acid; and wherein any residue except those in the P1, P4, and P5 pockets is replaced by a small neutral amino acid.

127. The peptide according to claim 126, wherein the small neutral amino acid is selected from the group consisting of alanine, serine and glycine residues.

128. The isolated peptide composition according to claim 126, wherein the amino acid sequence of the peptide further comprises at least one proline residue located at a position selected from the terminal and penultimate amino acid residues.

129. An isolated peptide composition that binds to a class II MHC protein HLA-DR2 and stimulates proliferation of Th2 secreting T cells.

130. The peptide according to claim 129, wherein the T cells secrete at least one cytokine selected from the group consisting of IL-4, IL-10 and IL-13.

131. A method for treating a demyelinating condition in a subject, comprising: administering to the subject a composition comprising at least one peptide selected from the group consisting of: EKAKEEAYKAAAAAA (SEQ ID NO:4), APEKAKVEAFKAAAAPA (SEQ ID NO: 10), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EKAKEEAYK (SEQ ID NO: 30), EKAKFEAYK (SEQ ID NO: 34), EKAKVEAFK (SEQ ID NO: 35), EAPKFEAYK (SEQ ID NO: 37), EAPKFEAFK (SEQ ID NO: 39), EAKKFEAFK (SEQ ID NO: 40), EAKKFEAYK (SEQ ID NO: 41), EEAYK (SEQ ID NO: 21), VEAFK (SEQ ID NO: 25), and YEAFK (SEQ ID NO: 26); and, observing prevention or reduction of symptoms of a demyelinating condition in the subject.

132. The method according to claim 131, wherein administering is preventing symptoms.

133. The method according to claim 132, wherein the subject is asymptomatic.

134. The method according to either according to claims 132 or 133, wherein the subject has an HLA-DR2 haplotype.

135. The method according to claim 134, wherein the haplotype is selected from the group consisting of DRA*0101, DRB1*1501 and alleles related to DRB1*1501, DRB1*1601 and alleles related to DRB1*1601, DQA1*0102, and DQB1*0602.

136. The method according to claim 131, wherein prior to administering, the method further comprises testing T cells from the subject for reactivity to a dominant autoimmune epitope for MS.

137. The method according to claims 136, wherein the epitope is a myelin basic protein (MBP) peptide.

138. The method according to claim 137, wherein the epitope is MBP 85-99.

139. The method according to claim 131, wherein the subject is a mammal.

140. The method according to claim 139, wherein the mammal is a mouse with experimental allergic encephalomyelitis.

141. The method according to claim 139, wherein the mammal is a humanized mouse.

142. The method according to claim 131, wherein the subject is a human.

143. The method according to claim 142, wherein the subject is a patient with MS.

144. The method according to claim 142, wherein treating or reducing is decreasing severity or frequency of recurrences of symptoms.

145. The method according to claim 131, wherein the peptide is administered by at least one route selected from the group of: bolus injection selected from the group of intravenous (i.v.), subcutaneous (s.c.), intramuscular (i.m.), and intraperitoneal (i.p.); intravenous infusion; and oral administration.

146. The method according to claim 145, further comprising after administering the composition, analyzing a physiological parameter of the demyelinating condition.

147. The method according to claim 146, wherein analyzing the physiological parameter is testing T cells from the subject for reactivity to a peptide of myelin basic protein.

148. The method according to claim 131, further comprising administering an additional therapeutic agent.

149. The method according to claim 148, wherein the additional therapeutic agent is at least one agent selected from the group consisting of: an antibody, an enzyme inhibitor, an antibacterial agent, an antiviral agent, a steroid, a nonsteroidal anti-inflammatory agent, an antimetabolite, a cytokine, a cytokine blocking agent, an adhesion molecule blocking agent, a soluble cytokine receptor, and a random linear amino acid copolymer composition.

150. The method according to claim 149, wherein the additional agent is at least one agent selected from the group of: the cytokine which is an interferon; the copolymer selected from the group of YEAK (Copaxone®), YAK, FYAK, VWAK and VFAK; and the immune suppressant which is a drug or a protein, wherein the drug is selected from the group of rapamycin and FK506, and the protein is OKT3.

151. A kit for treating a subject having a demyelinating condition comprising a peptide having a sequence of amino acids according to claim 97 and a container.

152. The kit according to claim 151, further comprising instructions for use.

153. The kit according to claim 151, comprising the peptide in a unit dose.

154. A method of inhibiting secretion of an interleukin or a cytokine by an HLA-DR-2-restricted T cell, the method comprising contacting the T cell with at least one composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41).

155. The method according to claim 154, wherein the interleukin or cytokine is interleukin-2 (IL-2) or gamma interferon (IFN-γ).

156. The method according to claim 154, wherein the interleukin is a non-Th2 interleukin.

157. The method according to claim 154, further comprising observing inhibition of secretion that is greater than an inhibition observed in cells contacted with a random amino acid polymer.

158. The method according to claim 156, further comprising observing stimulation of a Th2 interleukin.

159. The method according to claim 158, wherein the Th2 interleukin comprises interleukin-4 (IL-4) or interleukin-10 (IL-10).

160. A method of inhibiting expansion of an antigen-specific T cell comprising contacting the T cell with a composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO: 1), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41).

161. The method according to claim 160, further comprising observing that the contacted T cell fails to increase proliferating.

162. The method according to claim 161, wherein the T cell is specific to a demyelinating condition.

163. The method according to claim 161, wherein the autoimmune T cell is specific to multiple sclerosis.

164. The method according to claim 161, wherein the T cell is in a subject.

165. A method of ameliorating development of symptoms of a demyelinating condition in a subject, comprising administering to the subject a composition having an amino acid sequence selected from the group consisting of: EKAKYEAYKAAAAAA (SEQ ID NO: 1), EKPKYEAYKAAAAPA (SEQ ID NO: 2), EKPKFEAYKAAAAPA (SEQ ID NO: 3), EKAKEEAYKAAAAAA (SEQ ID NO: 4), EKPKVEAYKAAAAPA (SEQ ID NO: 5), EKPKEEAFKAAAAPA (SEQ ID NO: 6), EKAKFEAFKAAAAAA (SEQ ID NO: 7), APEKAKFEAFKAAAAPA (SEQ ID NO: 8), APEKAKFEAYKAAAAPA (SEQ ID NO: 9), APEKAKVEAFKAAAAPA (SEQ ID NO: 10), EAKKYEAYKAAAAAA (SEQ ID NO:11), EAPKFEAYKAAAAPA (SEQ ID NO: 12), EAPKVEAYKAAAAPA (SEQ ID NO: 13), EAPKFEAFKAAAAPA (SEQ ID NO: 14), APEAKKFEAFKAAAAPA (SEQ ID NO: 15), APEAKKFEAYKAAAAPA (SEQ ID NO: 16), and APEAKKVEAFKAAAAPA (SEQ ID NO: 17), EAKKVEAFK (SEQ ID NO: 18), YEAYK (SEQ ID NO: 19), FEAYK (SEQ ID NO: 20), EEAYK (SEQ ID NO: 21), VEAYK (SEQ ID NO: 22), EEAFK (SEQ ID NO: 23), FEAFK (SEQ ID NO: 24), VEAFK (SEQ ID NO: 25), and YEAFK (SEQ ID NO: 26), EKAKYEAYK (SEQ ID NO: 27), EKPKYEAYK (SEQ ID NO: 28), EKPKFEAYK (SEQ ID NO: 29), EKAKEEAYK (SEQ ID NO: 30), EKPKVEAYK (SEQ ID NO: 31), EKPKEEAFK (SEQ ID NO: 32), EKAKFEAFK (SEQ ID NO: 33), EKAKFEAYK (SEQ ID NO: 34), EKAKVEAFK (SEQ ID NO: 35), EAKKYEAYK (SEQ ID NO: 36), EAPKFEAYK (SEQ ID NO: 37), EAPKVEAYK (SEQ ID NO: 38), EAPKFEAFK (SEQ ID NO: 39), EAKKFEAFK (SEQ ID NO: 40), and EAKKFEAYK (SEQ ID NO: 41).

166. The method according to claim 165, wherein the subject is asymptomatic.

167. The method according to claim 166, wherein the subject carries an HLA-DR2 haplotype.

168. The method according to claim 167, wherein the haplotype comprises at least one allele selected from the group of: DRA*0101, DRB1*1501, DQA1*0102, and DQB1*0602.

169. The method according to claim 166, wherein the subject contains a greater number of T cells that react specifically with a myelin specific autoantigen.

170. The method according to claim 169, wherein the autoantigen is selected from MBP 85-99 and PLP 139-151.
